(19) United States
(12) Patent Application Publication
(10) Pub. No.: US 2025/0295611 A1
(43) Pub. Date: Sep. 25, 2025

(54) METHODS OF TREATING 5HT2A RECEPTOR-MEDIATED CONDITIONS

(71) Applicant: Tessellate Therapeutics Inc., Henderson, NV (US)

(72) Inventors: Dustin Hines; Rochelle Hines

(21) Appl. No.: 18/860,502

(22) PCT Filed: Apr. 27, 2023
(86) PCT No.: PCT/US23/20276

§ 371 Date: Oct. 25, 2024

Related U.S. Application Data
(60) Provisional application No. 63/335,688, filed on Apr. 27, 2022.

(51) Int. Cl. A61K 31/137; A61K 31/341; A61K 31/381; A61K 31/4418

(52) U.S. Cl. CPC A61K 31/137; A61K 31/341; A61K 31/381; A61K 31/4418

(57) ABSTRACT

Provided herein, inter alia, methods of treating or ameliorating a 5HT2A mediated condition in a subject, compositions (e.g., pharmaceutical compositions), and kits for use in treating or ameliorating a 5HT2A mediated condition in a subject.

### CROSS-REFERENCES TO RELATED APPLICATION

This application claims the benefit of priority of U.S. Provisional Application No. 63/335,688 filed on Apr. 27, 2022, which is incorporated herein by reference in its entirety and for all purposes.

### BACKGROUND

Receptors for serotonin (5-hydroxytryptamine, 5HT) are a class of G protein coupled receptors. Serotonin is thought to play a role in processes related to learning and memory, sleep, thermoregulation, mood, motor activity, pain, sexual and aggressive behaviors, appetite, neurodegenerative regulation, and biological rhythms. Serotonin is linked to pathophysiological conditions such as anxiety, depression, obsessive compulsive disorders, schizophrenia, suicide, autism, migraine, emesis, alcoholism, and neurodegenerative disorders.

The effects of serotonin are mediated by at least 15 genetically distinct 5HT receptor subtypes, have been identified and assigned to one of seven families (5HT1-7). Each subtype indicates a unique distribution, preference for various agonists and correlate or functional correlates. Many of these receptors, including subclass 5HT2A, are G-protein coupled receptors (GPCRs) that signal by activation of guanine nucleotide binding proteins (G proteins), which results in the generation or inhibition of second molecules.

The subtype of the 5HT2A receptor (also referred to as a subclass) is discreetly widely expressed in the human brain, including many cortical, limbic and post-brain regions that are postulated to be involved in the modulation of higher cognitive and affective functions. The 5HT2A receptor has been postulated to be associated with a variety of diseases and disorders such as hypertension, thrombosis, migraine, vasospasm, ischemia, depression, anxiety, psychosis, schizophrenia, sleep disorders and appetite disorders.

### SUMMARY

The disclosure provides, inter alia, compositions and methods for treatment of diseases and disorders associated with the 5HT2A receptor.

More particularly, in one aspect, we provide compositions and compositions for modulation of a subject's response to a 5HT2A receptor agonist.

Modulation of a response may include for example moderation (particularly reduction) of any hallucinogenic effects that may be experienced in a subject upon administration of a 5HT2A receptor agonist.

In one embodiment, the compositions and methods may include administration of therapeutic agent (5HT2A receptor modulator compound) in conjunction or combination with a 5HT2A receptor agonist. In certain aspects, a 5HT2A receptor modulator compound and a 5HT2A receptor agonist may be administered substantially simultaneously to a subject.

In certain other aspects, a 5HT2A receptor agonist may be first administered to a subject followed by administration of a 5HT2A receptor modulator compound, for example the 5HT2A receptor modulator compound may be administered 1, 2, 3, 4, 5, 6, 7, 8, 9, 10, 12, 14, 16, 18, 20, 22, 24, 30, 36 or 48 hours or more after administration of a 5HT2A receptor agonist to a subject. In other aspects, a 5HT2A receptor modulator compound may be first administered to a subject followed by administration of a 5HT2A receptor agonist. As referred to herein, each of these embodiments can be described as administration of a 5HT2A receptor agonist in combination or conjunction with administration of a 5HT2A receptor modulator compound.

In the present methods and compositions, a wide variety of 5HT2A receptor agonists may be used, including one or more of an ergoline compound, a tryptamine compound and/or a phenethylamine compound. In one aspect, 5HT2A receptor agonists for use in the present method and compositions include LSD ((lysergic acid diethylamide), psilocybin, psilocin, mescaline, and/or (±)-2,5-dimethoxy-4-iodoamphetamine hydrochloride (DOI).

In additional aspects, 5HT2A receptor agonists for use in the present method and compositions include indoleamine and phenethylamine hallucinogens.

In a yet further aspect, 5HT2A receptor agonists for use in the present method and compositions include compounds of the following Formula (I):

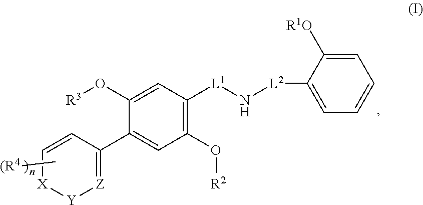

or pharmaceutically acceptable salt thereof,
wherein:
X, Y, and Z are each independently —C═ or —N═;
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl; and
n is an integer of 0 to 5.

In embodiments, in Formula (I), X is —C═, Y is —C═ and Z is —C═.

In certain embodiments, in Formula (I), X is —N═, Y is —C═ and Z is —C═.

In certain embodiments, in Formula (I), X is —N═, Y is —N═ and Z is —C═.

In certain embodiments, in Formula (I), X is —N═, Y is —C═ and Z is —N═.

In certain embodiments, in Formula (I), L¹ and L² are each independently substituted or unsubstituted C₁-C₄ alkylene.

In certain embodiments, in Formula (I), R¹, R², and R³ are each independently hydrogen, or substituted or unsubstituted C₁-C₄ alkyl.

In certain embodiments, in Formula (I), R¹ is hydrogen, and R² and R³ are each independently substituted or unsubstituted C₁-C₄ alkyl.

In certain embodiments, n is 0.

Exemplary preferred compounds of Formula (I) include the following compounds:

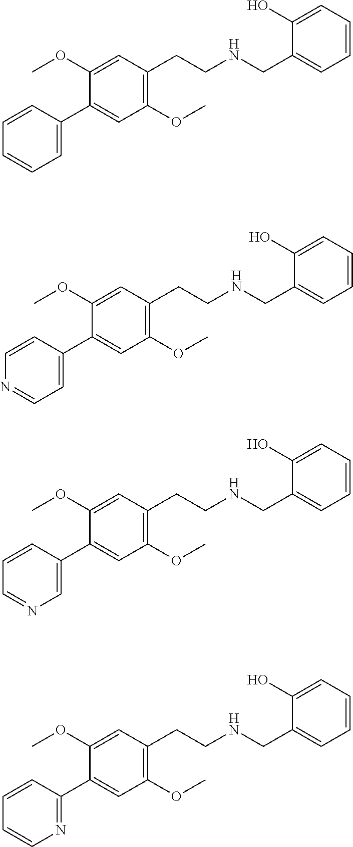 [SMILES: COc1cc(-c2ccccc2)c(OC)cc1CCNCc1ccccc1O ; COc1cc(-c2ccncc2)c(OC)cc1CCNCc1ccccc1O ; COc1cc(-c2cccnc2)c(OC)cc1CCNCc1ccccc1O ; COc1cc(-c2ccccn2)c(OC)cc1CCNCc1ccccc1O]

Further preferred 5HT2A receptor agonists for use in the present method and compositions are disclosed in U.S. Provisional Application No. 63/300,604 filed Jan. 18, 2022 and International Application No. PCT/US23/60844 filed Jan. 18, 2023.

In the present methods and compositions, 5HT2A receptor modulator compounds include: (a) alpha7 nicotinic acetylcholine receptor modulators; (b) alpha4 nicotinic acetylcholine receptor modulators; (c) monoaminergic system modulators; (d) cholesterol biosynthesis modulators; (e) cytokines/inflammatory modulators; (f) statin compounds; (g) immune system modulators and/or (h) trace amine-associated receptor modulators.

In an aspect, provided are methods and compositions that include administering: (i) one or more or 5HT2A receptor agonists to a subject, and (ii) one or more of therapeutic modulator agents, which are selected from (a) alpha7 nicotinic acetylcholine receptor modulators; (b) alpha4 nicotinic acetylcholine receptor modulators; (c) monoaminergic system modulators; (d) cholesterol biosynthesis modulators; (e) cytokines/inflammatory modulators; (f) statin compounds; (g) immune system modulators and/or (h) trace amine-associated receptor modulators.

In certain aspects, the one or more of therapeutic agents are administered before, or concurrently with, or subsequently after administration of the one or more or 5HT2A receptor agonists.

In certain aspects, the present methods are for treating or ameliorating altered patterns of brain activity in a subject.

In certain aspects, the present methods are for treating or ameliorating a 5HT2A receptor associated disease or disorder.

In certain aspects, the present methods are for treating or ameliorating a condition mediated by 5HT2A receptor activity. In certain aspects, the condition mediated by 5HT2A receptor activity is selected from psychotic disorders (such as schizophrenia), sleep disorders, depression, anxiety, panic disorder, post-traumatic stress disorder, obsessive-compulsive disorder, pain, eating disorders (such as anorexia nervosa), substance use disorder, dependency or acute toxicity associated with psychoactive agents such as LSD or MDMA, and hot flushes associated with the menopause.

In certain aspects, the present methods are for treating or ameliorating hallucinogenic symptoms or occurrences in a subject.

In certain aspects, the present methods and compositions may be used to treat a subject suffering from or susceptible to treatment resistant depression.

In one aspect, the present methods and compositions may be used to treat a subject suffering from or susceptible to post-traumatic stress disorder.

In one aspect, the present methods and compositions may be used to treat a subject suffering from or susceptible to substance use disorder.

In one aspect, the present methods and compositions may be used to treat a subject suffering from or susceptible to psychiatric disorder related sleep disturbances.

In certain aspects, the one or more 5HT2A receptor modulator compounds are administered after about 6 hours of administration of the one or more or 5HT2A receptor agonist compounds.

In certain aspects, the one or more 5HT2A receptor modulator compounds are administered after about 12 hours of administration of the one or more or 5HT2A receptor agonist compounds.

In certain aspects, the one or more 5HT2A receptor modulator compounds are administered after about 24 hours of administration of the one or more or 5HT2A receptor agonist compounds.

In certain aspects, the one or more 5HT2A receptor modulator compounds are administered after about 24 to 48 hours of administration of the one or more or 5HT2A receptor agonist compounds.

In certain aspects, the subject is a mammal. In some embodiments, the subject is a human.

In certain aspects, a preferred alpha7 nicotinic acetylcholine receptor modulator may include tropisetron, i.e. a compound of the following structure:

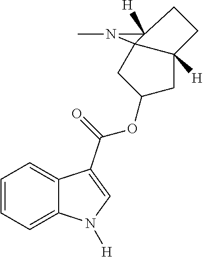 [SMILES: CN1[C@@H]2CC[C@@H]3CC(OC(=O)c4c[nH]c5ccccc45)CC321]

In certain aspects, a preferred alpha7 nicotinic acetylcholine receptor modulator may include WAY-317538 (SEN-12333), i.e. a compound of the following structure:

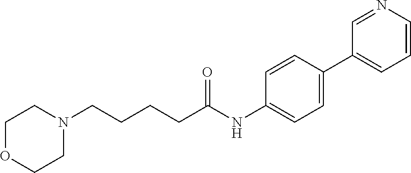 [SMILES: O=C(CCCCN1CCOCC1)Nc1ccc(-c2cccnc2)cc1]

In certain aspects, preferred immune system modulators may include antibodies and antibody fragments such as Gamifant® (emapalumab-lzsg) (interferon gamma (IFNγ)-blocking antibody).

In certain additional aspects, preferred immune system modulators may include tumor necrosis factor (TNF)-alpha inhibitors, including etanercept (E), infliximab (I), adalimumab (A), certolizumab pegol (C), and golimumab (G).

In certain additional aspects, preferred immune system modulators may include humanized anti-IL-9 monoclonal antibody, MEDI-528.

In further aspects, preferred statin compounds to use in the present methods and compositions include agents that may readily cross the blood-brain barrier, for example lipoholic statin agents. Specifically, preferred statin compounds include atorvastatin, lovastatin and simvastatin:

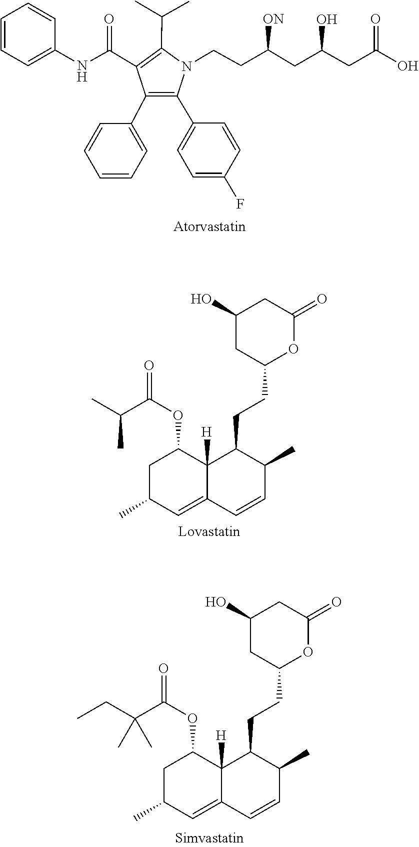

In certain aspect, the 5HT2A receptor agonists include a compound having a formula (X-a),

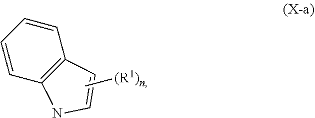

wherein
R¹ is hydrogen,
   halogen, —CN, —NR^{1A}R^{1B}, —C(O)R^{1C}, —C(O)—OR^{1C}, —C(O)NR^{1A}R^{1B}, —OR¹D, —NR^{1A}C (O)R^{1C}, —NR^{1A}C(O)OR^{1C}, —NR^{1A}OR^{1C}, —OR^{1D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is 1, or 2; and
R^{1A}, R^{1B}, R^{1C}, and RD are each independently hydrogen, substituted or unsubstituted alkyl, or substituted or unsubstituted heteroalkyl.

In some embodiments, R¹ is amine-substituted alkyl (e.g., C₁-C₃ alkyl). In certain embodiments, R¹ is —CH₂—CH₂—NH₂.

Exemplary 5HT2A receptor agonists may include, but not limited to, indolamines having a structure of

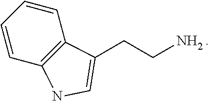 [SMILES: NCCc1c[nH]c2ccccc12]

In certain aspect, the 5HT2A receptor agonists include a compound having a formula (X-b),

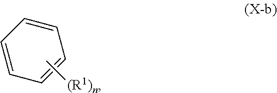

wherein n is an integer from 1 to 5. R¹ may be as described above.

In certain embodiments, R¹ is amine-substituted alkyl (e.g., C₁-C₃ alkyl). In certain embodiments, R¹ is —CH₂—CH₂—NH₂.

Exemplary 5HT2A receptor agonists may include, but not limited to, indolamines having a structure of

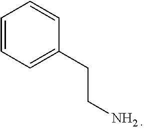

In certain aspects, the 5HT2A receptor modulator compounds are alpha7 nicotinic acetylcholine receptor modulators may include one or more selected from: (+)-N-(1-azabicyclo[2.2.2]oct-3-yl)benzo[b]furan-2-carboxamide, tilorone, A-582941, AR-R17779, TC-1698, bradanicline, encenicline, GTS-21, PHA-543,613, PNU-282,987, PHA-709829, SSR-180,711, tropisetron, WAY-317,538, anabasine, acetylcholine, nicotine, epiboxidine, choline, ICH-3, PNU-120,596, NS-1738, AVL-3288, A-867744, ivermectin, nefiracetam, anandamide, α-bungarotoxin, α-conotoxin ArIB, β-caryophyllene, bupropion, dehydronorketamine, ethanol, hydroxybupropion, hydroxynorketamine, ketamine, kynurenic acid, memantine, methylcaconitine, mecamylamine, lobeline, methyllycaconitine, norketamine, and quinolizidine, dextromethorphan, amantadine, and derivatives thereof.

In certain aspects, the alpha4 nicotinic acetylcholine receptor modulators comprise one or more selected from: α-conotoxin, Dextromethorphan, dihydro-β-erythroidine, mecamylamine, bupropion, 3-Bromocytisine, Acetylcholine, Cytisine, Galantamine, Epibatidine, Epiboxidine, Nicotine, A-84,543, A-366,833, ABT-418, Arecoline, Altinicline, Dianicline, Ispronicline, Pozanicline, Rivanicline, Tebanicline, TC-1827, Varenicline, Sazetidine A, N-(3-pyridinyl)-bridged bicyclic diamines, NS-9283, Desformylflustrabromine, (−)-7-methyl-2-exo-[3′-(6-[18F]fluoropyridin-2-yl)-5′-pyridinyl]-7-azabicyclo[2.2.1]heptane, 2-fluoro-3-(4-nitro-phenyl)deschloroepibatidine, Coclaurine, Mecamylamine, α-Conotoxin, PNU-120,596, Bupropion, Dihydro-β-erythroidine, Nitrous oxide, Isoflurane, 1-(6-(((R,S)-7-Hydroxychroman-2-yl)methylanino]hexyl)-3-((S)-1-methylpyrrolidin-2-yl)pyridinium bromide, Oxantel, and derivatives thereof.

In certain aspects, the monoaminergic system modulators comprise one or more selected from 8-OH-DPAT, Adatanserin, Amphetamine, etoperidone, hydroxynefazodone, nefazodone, trazodone, triazoledione, vilazodone, vortioxetine, aripiprazole, asenapine, brexpiprazole, cariprazine, clozapine, lurasidone, quetiapine, ziprasidone, buspirone, eptapirone, gepirone, perospirone, tandospirone, Bay R 1531, Befiradol, BMY-14802, Cannabidiol, Dimemebfe, Dopamine, Ebalzotan, Eltoprazine, Enciprazine, bromocriptine, cabergoline, dihydroergotamine, ergotamine, lisuride, LSD, methylergometrine (methylergonovine), methysergide, pergolide, F-11461, F-12826, F-13714, F-14679, F-15063, F-15,599, Flesinoxan, Flibanserin, Flumexadol, Hypidone, Lesopitron, LY-293284, LY-301317, Naluzotan, NBUMP, Osemozotan, Oxaflozane, Pardoprunox, Piclozotan, Rauwolscine Repinotan, Roxindole, RU-24, 969, 5-14,506, S-14671, S-15535, Sarizotan, Serotonin (5-HT), SSR-181507, Sunepitron, 5-CT, 5-MeO-DMT, 5-MT, bufotenin, DMT, indorenate, N-Me-5-HT, psilocin, psilocybin, TGBA01AD, U-92,016-A, Urapidil, Vilazodone, Xaliproden, Yohimbine, iloperidone, risperidone, sertindole, AV965, alprenolol, arteolol, cyanopindolol, iodocyanopindolol, oxprenolol, penbutolol, pindobind, pindolol, propranolol, tertatolol, BMY-7,378, CSP-2503, Dotarizine, metergoline, FCE-24379, Flopropione, GR-46611, Lecozotan, Mefway, Metitepine (methiothepin), MIN-117 (WF-516), MPPF, NAN-190, Robalzotan, S-15535, SB-649,915, SDZ 216-525, Spiperone, Spiramide, Spiroxatrine, UH-301, WAY-100135, WAY-100635, Xylamidine, Acetryptine, Carvedilol, Ergolines (e.g., ergometrine (ergonovine)), Anpirtoline, CGS-12066A, CP-93129, CP-94253, CP-122,288, CP-135807, mCPP, RU-24,969, Vortioxetine, AR-A000002, carteolol, oxprenolol, penbutolol, propranolol, tertatolol, GR-127935, Isamoltane, LY-393558, SB-216641, SB-224289, SB-236057, CP-286601, L-694247, L-772405, PNU-109291, PNU-142633, Alniditan, BRL-15,572, Elzasonan, GR-127935, LY-310762, LY-367642, LY-456219, LY-456220, Mianserin, BRL-54443, Adatanserin, Altanserin, cyproheptadine, hydroxyzine, ketotifen, perlapine, AMDA, amperozide, aripiprazole, asenapine, blonanserin, brexpiprazole, carpipramine, clocapramine, clorotepine, clozapine, fluperlapine, gevotroline, lurasidone, melperone, mosapramine, ocaperidone, olanzapine, paliperidone, quetiapine, zicronapine, zotepine, Chlorprothixene, Cinanserin, CSP-2503, Deramciclane, Dotarizine, Eplivanserin, amesergide, LY-53857, LY-215,840, mesulergine, metergoline, methysergide, sergolexole, Fananserin, Flibanserin, Glemanserin, Irindalone, Ketanserin, KML-010, Landipirdine, Medifoxamine, MIN-117 (WF-516), Naftidrofuryl, Nantenine, Nelotanserin, Opiranserin (VVZ-149), Pelanserin, Phenoxybenzamine, Pimavanserin, Pirenperone, Pizotifen, Pruvanserin, Rauwolscine, Roluperidone, Sarpogrelate, lubazodone, mepiprazole, TGBAOTAD, Teniloxazine, Temanogrel, amoxapine, aptazapine, esmirtazapine, maprotiline, mianserin, mirtazapine, amitriptyline, chlorpromazine, fluphenazine, haloperidol, loxapine, perphenazine, pimozide, pipamperone, prochlorperazine, setoperone, spiperone, spiramide, thioridazine, thiothixene, trifluoperazine, Volinanserin, dihydroergotamine, nicergoline, 4-Methylaminorex, Aminorex, chlorphentermine, cloforex, dexfenfluramine, enfluramine, levofenfluramine, norfenfluramine, BW-723C86, cabergoline, dihydroergocryptine, dihydroergotamine, ergotamine, Lorcaserin, PNU-22394, Ro60-0175, Agomelatine, EGIS-7625, LY-393558, Metadoxine, PRX-08066, Ritanserin, RS-127445, SB-200646, SB-204741, SB-206553, SB-215505, SB-221284, SB-228357, SDZ SER-082, Tegaserod, and naphthylpiperazine.

In certain aspects, the cholesterol biosynthesis modulators comprise one or more selected from statins, lovastatin, simvastatin, pravastatin, fluvastatin, atorvastatin, cerivastatin, and TSPO agonists comprising YL-IPA08, Ro5-4864, Anthralin, Alpidem, DAA-1097, DAA-1106, DPA-713, DPA-714, Emapunil, Etifoxine, FGIN-127, FGIN-143, GML-1, SSR-180,575 or PK-11195.

In certain aspects, the cytokines/inflammatory modulators comprise one or more selected from CCL4 (MIP-1β), CCL5 (RANTES), CCL6, CCL9 (CCL10), CCL14, CCL15, CCL16, CCL23, CCL2, CCL8, CCL12, CCL16, Cenicriviroc (TAK-652, TBR-652). CCL5 (RANTES), CCL7, CCL11, CCL13, CCL15, CCL18, CCL24, CCL26, CCL28, CCL3 (MIP-1α), CCL5 (RANTES), CCL17, CCL22, Mogamulizumab, CCL3 (MIP-1α), CCL4 (MIP-1β), CCL5 (RANTES), CCL8, CCL11, CCL13, CCL14, CCL16, Aplaviroc, Cenicriviroc (TAK-652, TBR-652), INCB009471, Maraviroc, Vicriviroc, PRO-140 antibody, CCL20, CCL19, CCL21, CCL1, CCL16, CCL25, CCL27, CCL28, CCL19, CCL21, CCL25, Bertilimumab, Carlumab, CXCL6, Emoctakin, Interleukin-8 (CXCL8, GCP-1), Navarixin, Ladarixin, Reparixin (repertaxin), CXCL1 (MGSA), CXCL2, CXCL3, CXCL5, CXCL6, CXCL7, Emoctakin, Garnocestim, Interleukin-8 (CXCL8, GCP-1), Danirixin, Elubrixin, Navarixin, Ladarixin, Reparixin (repertaxin), CXCL4 (PF4), CXCL9 (MIG), CXCL10 (IP-10), CXCL11 (1-TAC), Iroplact, Eldelumab, MIF, SDF-1 (CXCL12), Ubiquitin, Mavorixafor, Plerixafor (AMD3100), Ulocuplumab, CXCL13, CXCL16, CXCL11 (I-TAC), SDF-1 (CXCL12), Plerixafor (AMD3100), Lymphotactin-α (XCL1), Lymphotactin-β (XCL2), Pateclizumab, Fractalkine (CX3CL1), CC(β) chemokines, Chemerin, Resolvin E1, ARA-290, Asialo erythropoietin, Carbamylated erythropoietin, CNTO-530, Darbepoetin alpha, Epoetin alpha, Epoetin beta, Epoetin delta, Epoetin epsilon, Epoetin gamma, Epoetin kappa, Epoetin omega, Epoetin theta, Epoetin zeta, Erythropoietin (EPO), Erythropoietin-Fc, Methoxy polyethylene glycol-epoetin beta (CERA/Mircera), Peginesatide, Pegol sihematide (EPO-018B), Filgrastim, Granulocyte colony-stimulating factor, Lenograstim, Leridistim, Lipegfilgrastim, Nartograstim, Pegfilgrastim, Pegnartograstim, Ecogramostim, Granulocyte macrophage colony-stimulating factor, Milodistim, Molgramostim, Regramostim, Sargramostim, Mavrilimumab, Namilumab, Otilimab, Cilmostim, Interleukin-34, Lanimostim, Macrophage colony-stimulating factor, Mirimostim, Agerafenib, Eltrombopag, Pegacaristim, Promegapoietin, Romiplostim, Thrombopoietin (THPO, MGDF), Albinterferon, Interferon alpha (interferon alpha, IFN-α), Interferon alpha (IFNA1, IFNA2, IFNA4, IFNA5, IFNA6, IFNA7, IFNA8, IFNA10, IFNA13, IFNA14, IFNA16, IFNA17, IFNA21), Interferon alpha 2a, Interferon alpha 2b, Interferon alpha n1, Interferon alphacon-1, Interferon alpha-n3, Interferon beta (IFN-β) (IFNB1, IFNB3), Interferon beta 1a, Interferon beta 1b, Interferon kappa (IFN-ε/κ/τ/ζ, IFNK), Interferon omega (IFN-ω, IFNW1), Peginterferon alpha-2a, Peginterferon alpha-2b, Anifrolumab, Faralimomab, MEDI-545, Rontalizumab, Sifalimumab, Bifarcept, Interferon gamma (IFN-γ), Interferon gamma 1b, Emapalumab, Fontolizumab, Interleukin 1 (α, β), Mobenakin, Pifonakin, AF-12198, Anakinra, IL-1RA, Isunakinra, Canakinumab, Gevokizumab, Lutikizumab, Decoy receptors: Rilonacept (IL-1 Trap), Adargileukin alpha, Aldesleukin, Celmoleukin, Denileukin diftitox, Interleukin 2, Pegaldesleukin, Teceleukin, Tucotuzumab celmoleukin, Basiliximab, Daclizumab (dacliximab), Inolimomab, Daniplestim, Interleukin 3, Leridistim, Milodistim, Muplestim, Promegapoietin, Binetrakin, Interleukin 4, Interleukin 13, Pitrakinra, Dupilumab, Pascolizumab, Interleukin 5, YM-90709, Benralizumab, Mepolizumab, Reslizumab, TPI ASM8, Atexakin alpha, Interleukin 6, ARGX-109, Clazakizumab, Elsilimomab, mAb 1339, Olokizumab, Sarilumab, Siltuximab, Sirukumab, Tocilizumab, Levilimab: Interleukin 7, Interleukin 9, Enokizumab, Ilodecakin, Interleukin 10 (CSIF), Interleukin 11 (AGIF), Oprelvekin, Edodekin alpha, Interleukin 12, Briakinumab, Ustekinumab, Binetrakin, Cintredekin besudotox, Interleukin 4, Interleukin 13, Anrukinzumab, Lebrikizumab, Tralokinumab, ALT-803, Interleukin 15, Interleukin 17 (A, B, C, D, E (interleukin 25), Brodalumab, Ixekizumab, Perakizumab, Remtolumab, Secukinumab, Vunakizumab, Iboctadekin, Interleukin 18, Interleukin 37, Tadekinig, IL18BP, Interleukin 19, Interleukin 20, Interleukin 24, Fletikumab, Denenicokin, Interleukin 21, NNC0114-0005, NNC0114-0006, Interleukin 22, Fezakinumab, Interleukin 23 (SGRF), Brazikumab, Briakinumab, Guselkumab, Risankizumab, Tildrakizumab, Ustekinumab, Interleukin 27 (interleukin 30), Interferon λ4 (IFN-λ4), Interleukin 28 (A (IFN-λ2), B (IFN-λ3)), Interleukin-29 (IFN-λ1), Interleukin 31, Interleukin 33, Interleukin 36 (α, β, γ), Interleukin 38, IL-36RA, Interleukin 14 (taxilin alpha, HMW-BCGF), Interleukin 16, Interleukin 24, Interleukin 26, Interleukin 32, Interleukin 34, Interleukin 35, Efavaleukin alpha, Efineptakin alpha, Activin (A, B, AB), Avotermin, BMP (10), Cetermin, GDF (2 (BMP9)), TGFβ (1, 2, 3): DMH-1, DMH-2, Dorsomorphin (BML-275), K-02288, ML-347 (LDN-193719, VU0469381), Ascrinvacumab, K-02288, ML-347 (LDN-193719, VU0469381), Dalantercept, Disitertide, Activin (A, B, AB), AMH (MIS), Avotermin, BMP (5, 6, 7, 8A, 8B), Eptotermin alpha, TGFβ (1, 2, 3), AMH (MIS), BMP (2, 4, 5, 6, 7, 8A, 8B), Dibotermin alpha, Eptotermin alpha, DMH-2, Dorsomorphin (BML-275), K-02288, Activin (A, B, AB), GDF (1, 3, 11 (BMP11)), Myostatin (GDF8), Nodal, Inhibin (A, B), Lefty (1, 2), A 83-01, SB-431542, SB-505124, Avotermin, GDF (10 (BMP3B), 11 (BMP11)), TGFβ (1, 2, 3), Fresolimumab, Lerdelimumab, Metelimumab, A 83-01, D-4476, GW-788388, LY-364947, LY-2109761, Galunisertib (LY-2157299), R-268712, RepSox (E-616452, SJN-2511), SB-431542, SB-505124, SB-525334, SD-208, BMP (2, 4, 5, 6, 7, 8A, 8B, 15, GDF9B)), BAMBI, Cerberus (CER1), Chordin, DAN (PARN), Decorin, Follistatin, Gremlin (Drm), LTBP1, Noggin, TGIF, Thrombospondin 1 (THBS1), Tomoregulin 1, Stamulumab, TRC105, Endoglin, Lymphotoxin, Baminercept, Plusonermin, Sonermin, Tasonfermin, Afelimomab, Certolizumab pegol, Golimumab, Infliximab, Nerelimomab, Ozoralizumab, Remtolumab, Placulumab, Belimumab, Brentuximab vedotin, Conatumumab, Dacetuzumab, Denosumab, Drozitumab, Enavatuzumab, Iratumumab, Lexatumumab, Lucatumumab, Mapatumumab, Oxelumab, Ruplizumab, Tabalumab, Tavolixizumab, Teneliximab, Tigatuzumab, Toralizumab, Urelumab, Utomilumab, Varlilumab, Vorsetuzumab, Vorsetuzumab mafodotin, Abrocitinib, Baricitinib. Filgotinib. Momelotinib, Oclacitinib, Peficitinib, Ruxolitinib, Tofacitinib (tasocitinib, CP-690550), Upadacitinib, Atiprimod, AZD-1480, Baricitinib, CHZ868, Cucurbitacin I (elatericin B, JSI-124), CYT387, Lestaurtinib, NSC-7908, NSC-33994, Pacritinib, Peficitinib, Ruxolitinib, SD-1008, Tofacitinib (tasocitinib, CP-690550), Cercosporamide, Decernotinib (VX-509), Peficitinib, TCS-21311, Tofacitinib (tasocitinib, CP-690550), WHI-P 154, ZM-39923, ZM-449829, Cardiotrophin 1 (CT-1), FMS-like tyrosine kinase 3 ligand (FLT3L), Leukemia/leukocyte inhibitory factor (LIF), Oncostatin M (OSM), Thymic stromal lymphopoietin (TSLP), Lestaurtinib, Midostaurin, Quizartinib, Sorafenib, and Sunitinib.

In certain aspects, the trace amine-associated receptor modulators comprise one or more selected from SEP-363856, RO5166017, RO5256390, RO5203648, RO5263397, RO5073012, and o-PIT.

The present therapeutic methods and use include treating a subject suffering from a psychotic disorder, sleep disorder, depression, anxiety, panic disorder, obsessive-compulsive disorder, post-traumatic stress disorder, substance use disorder, pain, eating disorder, dependency or acute toxicity associated with narcotic agents such as LSD or MDMA, and/or hot flushes associated with the menopause.

In certain aspects, 1) the subject is identified as suffering from a psychotic disorder, sleep disorder, depression, anxiety, panic disorder, obsessive-compulsive disorder, post-traumatic stress disorder, substance use disorder, pain, eating disorder, dependency or acute toxicity associated with narcotic agents such as LSD or MDMA, and or hot flushes associated with the menopause and 2) the (i) one or more or 5HT2A receptor agonists, and (ii) one or more 5HTA receptor modulator compounds are administered to the identified subject.

The present therapeutic methods and use also include treating a subject is suffering from hallucinogenic symptoms or occurrences. In certain aspects, 1) the subject is identified as suffering from hallucinogenic symptoms or occurrences and 2)) the (i) one or more or 5HT2A receptor agonists, and (ii) one or more 5HTA receptor modulator compounds are administered to the identified subject.

The present therapeutic methods and use also include treating a subject is suffering from or susceptible to treatment resistant depression. In certain aspects, 1) the subject is identified as suffering from or susceptible to treatment resistant depression and 2) the (i) one or more or 5HT2A receptor agonists, and (ii) one or more 5HTA receptor modulator compounds are administered to the identified subject.

The present therapeutic methods and use also include treating a subject suffering from post-traumatic stress disorder. In certain aspects, 1) the subject is identified as suffering from post-traumatic stress disorder and 2) the (i) one or more or 5HT2A receptor agonists, and (ii) one or more 5HTA receptor modulator compounds are administered to the identified subject.

The present therapeutic methods and use also include treating a subject suffering from or susceptible to substance use disorder. In certain aspects, 1) the subject is identified as suffering from or susceptible to substance use disorder and 2) the (i) one or more or 5HT2A receptor agonists, and (ii) one or more 5HTA receptor modulator compounds are administered to the identified subject.

In an aspect, provided is a kit for treating or ameliorating altered patterns of brain activity in a subject. The kit includes: (i) one or more or 5HT2A receptor agonist compounds, and (ii) one or more of therapeutic agents, which are selected from (a) alpha7 nicotinic acetylcholine receptor modulators; (b) alpha4 nicotinic acetylcholine receptor modulators; (c) monoaminergic system modulators; (d) cholesterol biosynthesis modulators; (e) cytokines/inflammatory modulators; (f) statin compounds; (g) immune system modulators and/or (h) trace amine-associated receptor modulators. A kit suitably further comprises instructions for treating a disease or disorder associated with the 5HT2A receptor, e.g. written instructions such as a packaging insert or product label.

In certain aspects, the one or more of therapeutic agents are contained in a composition mixed or combined with the one or more 5HT2A receptor agonist compounds. In certain aspects, the one or more of therapeutic agents and the one or more or 5HT2A receptor agonist compounds are contained in separate compositions.

Other aspects of the invention are disclosed infra.

### DETAILED DESCRIPTION OF THE INVENTION

Provided herein, inter alia, methods of treating or ameliorating altered patterns of brain activity in a subject, compositions (e.g., pharmaceutical compositions), and kits for use in treating or ameliorating altered patterns of brain activity in a subject.

### I. Definitions

As used herein, the term “a,” “an,” “the” and similar terms used in the context of the present invention (especially in the context of the claims) are to be construed to cover both the singular and plural unless otherwise indicated herein or clearly contradicted by the context.

The language “and/or” is used herein as a shorthand notation to represent the expression “and,” describing the combination of items, as well as “or,” describing the items in the alternative form.

As used herein, the term “treatment-resistant” means a lack of therapeutic response after at least one trial of an antidepressant at an adequate dose for six weeks.

The term “compound” when referring to a compound of this invention, refers to a collection or population of molecules having an identical chemical structure, except that there may be isotopic variation among the constituent atoms of the molecules.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3).

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S.

The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle.

The term “alkylene,” by itself or as part of another substituent, means, unless otherwise stated, a divalent radical derived from an alkyl, as exemplified, but not limited by, —CH₂CH₂CH₂CH₂— Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si. or P) may be placed at any interior position of the heteroalkyl group or at the position at which the alkyl group is attached to the remainder of the molecule. Heteroalkyl is an uncyclized chain.

The term “heteroalkenvl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. A “cycloalkylene” and a “heterocycloalkylene,” alone or as part of another substituent, means a divalent radical derived from a cycloalkyl and heterocycloalkyl, respectively.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring.

The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring).

The term “substituted” means to contain or include at least one “substituent group”. For example, the “substituent group,” as used herein, means a group selected from oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N3, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C5-C6 cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, as within a range of normal tolerance in the art, for example, within 2 standard deviations of the mean. In certain embodiments, “about” can be understood as within 10%, 9%, 8%, 7%, 6%, 5%, 4%, 3%, 2%, 1%, 0.5%, 0.1%, 0.05%, or 0.01% of the stated value.

When a range is described for a variable, it will be understood that the variable includes all values including the end points described within the stated range. For example, the range of “5 to 10” will be understood to include any subranges, such as 6 to 10, 7 to 10, 6 to 9, 7 to 9, and the like, as well as individual values of 5, 6, 7, 8, 9 and 10, and will also be understood to include any value between valid integers within the stated range, such as 5.5, 6.5, 7.5, 5.5 to 8.5, 6.5 to 9, and the like. Also, for example, the range of “10% to 30%” will be understood to include subranges, such as 10% to 15%, 12% to 18%, 20% to 30%, etc., as well as all integers including values of 10%, 11%, 12%, 13% and the like up to 30%, and will also be understood to include any value between valid integers within the stated range, such as 10.5%, 15.5%, 25.5%, and the like.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)-isomers, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

The term “about”, as used herein, means an acceptable margin of error for a particular value, which depends in part on how the value is measured or determined. In certain embodiments, “about” as used herein will be understood by persons of ordinary skill in the art to mean up to plus or minus 20% of the particular term. In further embodiments, “about” as used herein will be understood by persons of ordinary skill in the art to mean up to plus or minus 10% of the particular term.

The term “isolated” as used herein, means having been removed from or is free of other compounds or biological and/or chemical materials present when the compound is first formed. The term “isolated” embraces compounds isolated from natural sources as well as chemically-synthesized compounds.

As used herein, the term “substantially pure” means sufficiently homogeneous to appear free of readily detectable impurities as determined by standard analytical methods, such as thin layer chromatography (TLC), gel electrophoresis, high performance liquid chromatography (HPLC), nuclear magnetic resonance (NMR), and mass spectrometry (MS); or sufficiently pure such that further purification would not detectably alter the physical and chemical properties, or biological and pharmacological properties, such as enzymatic and biological activities, of the substance. In certain embodiments, “substantially pure” refers to a collection of molecules, wherein at least about 55%, about 60%, about 65%, about 70%, about 75%, about 80%, about 85%, about 90%, about 95%, about 97%, about 98%, about 98.5%, about 99%, about 99.5% or about 99.9% or greater of the molecules are a single compound, including a racemic mixture or a single stereoisomer thereof, as determined by standard analytical methods.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it.

Similarly, cachets and lozenges are included. Tablets, powders, capsules, pills, cachets, and lozenges can be used as solid dosage forms suitable for oral administration.

“Contacting” is used in accordance with its plain ordinary meaning and refers to the process of allowing at least two distinct species (e.g. chemical compounds including biomolecules or cells) to become sufficiently proximal to react, interact or physically touch.

It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

As used herein, and unless otherwise specified, the terms “treat,” “treating” and “treatment” refer to the eradication or amelioration of a disease, disorder, or condition, or of one or more symptoms associated with the disease, disorder or condition. In certain embodiments, the terms refer to minimizing the advancement or worsening of the disease, disorder, or condition resulting from the administration of a formulation of the invention to a patient with such a disease, disorder, or condition. In some embodiments, the terms refer to the administration of a formulation provided herein, after the onset of symptoms of the particular disease, disorder, or condition. The terms “treat,” “treating”, “treatment”, or the like, as used herein covers the treatment of a disease, disorder, or condition in a subject, e.g., a mammal, and includes at least one of: (i) inhibiting the disease, disorder, or condition, i.e., partially or completely halting its progression; (ii) relieving the disease, disorder, or condition, i.e. causing regression of symptoms of the disease, disorder, or condition, or ameliorating a symptom of the disease, disorder, or condition; and (iii) reversal or regression of the disease, disorder, or condition, preferably eliminating or curing of the disease, disorder, or condition. In a particular embodiment the terms “treat,” “treating”, “treatment”, or the like, covers the treatment of a disease, disorder, or condition in a mammal, e.g., a primate, e.g., a human, and includes at least one of (i), (ii), and (iii) above. As is known in the art, adjustments for age, body weight, general health, sex, diet, time of administration, drug interaction and the severity of the condition may be necessary, and will be ascertainable with routine experimentation by one of ordinary skill in the art based on the invention described herein.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

As used herein, the terms “subject”, and “patient” are used interchangeably. The terms “subject” and “patient” refer to an animal (e.g., a bird such as a chicken, quail or turkey) or a mammal including non-primates (e.g., a cow, pig, horse, sheep, rabbit, guinea pig, rat, cat, dog, and mouse) and primates (e.g., a monkey, chimpanzee and a human). In a particular embodiment, the subject is a human.

As used herein, and unless otherwise specified, the terms “prevent,” “preventing” and/or “prevention” refer to the prevention of the onset, recurrence or spread of a disease, disorder, or condition, or of one or more symptoms thereof. In certain embodiments, the terms refer to the administration of a compound of any one of Formulae (I), (II) and/or (III), or a pharmaceutically acceptable salt thereof, to a subject, with or without other additional active compounds, prior to the onset of symptoms, particularly to patients at risk of a disease, disorder, or condition provided herein. The terms encompass the inhibition or reduction of a symptom of the particular disease, disorder, or condition. Subjects with familial history of a disease, disorder, or condition, in particular, are candidates for preventive regimens in certain embodiments. In addition, subjects who have a history of recurring symptoms are also potential candidates for the prevention. In this regard, the terms “prevent,” “preventing” and/or “prevention” may be interchangeably used with the term “prophylactic treatment.”

As used herein, and unless otherwise specified, a “therapeutically effective amount” or an “effective amount” of an active agent of a compound of any one of Formulae (I), (II) and/or (III) or a pharmaceutically acceptable salt thereof, is an amount sufficient to provide a therapeutic benefit in the treatment or management of a disease, disorder, or condition, or to delay or minimize one or more symptoms associated with the disease, disorder, or condition The term “therapeutically effective amount” can encompass an amount that improves overall therapy, reduces or avoids symptoms or causes of disease, disorder, or condition, or enhances the therapeutic efficacy of another therapeutic agent. The therapeutically effective amount for a particular patient in need of such treatment can be determined by considering various factors, such as the condition treated, the overall health of the patient, method of administration, the severity of side-effects, and the like. For any compound described herein, the therapeutically effective amount can be initially determined from cell culture assays. Target concentrations will be those concentrations of active compound(s) that are capable of achieving the methods described herein, as measured using the methods described herein or known in the art. As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

As used herein, and unless otherwise specified, the terms “manage,” “managing” and “management”, are used interchangeably herein and refer to preventing or slowing the progression, spread or worsening of a disease, disorder, or condition, or of one or more symptoms thereof. Often, the beneficial effects that a subject derives from a prophylactic and/or therapeutic agent do not result in a cure of the disease, disorder, or condition. In this regard, the terms “manage,” “managing” and/or “management”, encompass treating a subject who had suffered from the particular disease, disorder, or condition in an attempt to prevent or minimize the recurrence of the disease, disorder, or condition.

“Patient” or “subject” or “subject in need thereof” refers to a living organism or animal diagnosed with, suffering from or prone to a disease, disorder or condition that can be treated by administration of a compound as disclosed herein. Non-limiting examples include humans, other mammals, bovines, rats, mice, dogs, monkeys, goat, sheep, cows, deer, and other non-mammalian animals. In some embodiments, a patient is human.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

### II. Methods

In an aspect, provided are methods of treating or ameliorating altered patterns of brain activity in a subject. The method includes administering a subject (i) one or more or 5HT2A (5-hydroxy-tryptamine) receptor agonists, and (ii) one or more of therapeutic modulator agents. In certain aspect, the one or more therapeutic agents include one or more selected from (a) alpha7 nicotinic acetylcholine receptor modulators; (b) alpha4 nicotinic acetylcholine receptor modulators; (c) monoaminergic system modulators; (d) cholesterol biosynthesis modulators; and (e) cytokines/inflammatory modulators.

In certain aspects, the one or more of therapeutic agents are administered concurrently with or subsequently after administration of the one or more or 5HT2A (5-hydroxy-tryptamine) receptor agonists. In some embodiments, the one or more of therapeutic agents are administered concurrently with administration of the one or more or 5HT2A (5-hydroxy-tryptamine) receptor agonists. In some embodiments, the one or more of therapeutic agents are administered subsequently after administration of the one or more or 5HT2A (5-hydroxy-tryptamine) receptor agonists.

In certain aspect, the one or more of therapeutic agents are administered within about 1 hours to about 72 hours after administration of the one or more or 5HT2A receptor agonists. In some embodiments, the one or more of therapeutic agents are administered within about 1 hours to about 48 hours after administration of the one or more or 5HT2A receptor agonists. In some embodiments, the one or more of therapeutic agents are administered within about 1 hours to about 24 hours after administration of the one or more or 5HT2A receptor agonists. In some embodiments, the one or more of therapeutic agents are administered within about 24 hours to about 72 hours after administration of the one or more or 5HT2A receptor agonists. In some embodiments, the one or more of therapeutic agents are administered within about 24 hours to about 60 hours after administration of the one or more or 5HT2A receptor agonists. In some embodiments, the one or more of therapeutic agents are administered within about 24 hours to about 48 hours after administration of the one or more or 5HT2A receptor agonists.

The compounds (e.g., the one or more of therapeutic agents) provided herein can be administered alone or can be coadministered to the subject. Coadministration is meant to include simultaneous or sequential administration of the compounds individually or in combination (more than one compound). Thus, the preparations can also be combined, when desired, with other active substances (e.g. to reduce metabolic degradation).

In some embodiments, the one or more of therapeutic agents are administered after about 1 hours of administration of the one or more or 5HT2A receptor agonists. In some embodiments, the one or more of therapeutic agents are administered after about 2 hours of administration of the one or more or 5HT2A receptor agonists. In some embodiments, the one or more of therapeutic agents are administered after about 3 hours of administration of the one or more or 5HT2A receptor agonists. In some embodiments, the one or more of therapeutic agents are administered after about 4 hours of administration of the one or more or 5HT2A receptor agonists. In some embodiments, the one or more of therapeutic agents are administered after about 5 hours of administration of the one or more or 5HT2A receptor agonists. In some embodiments, the one or more of therapeutic agents are administered after about 6 hours of administration of the one or more or 5HT2A receptor agonists. In some embodiments, the one or more of therapeutic agents are administered after about 7 hours of administration of the one or more or 5HT2A receptor agonists. In some embodiments, the one or more of therapeutic agents are administered after about 8 hours of administration of the one or more or 5HT2A receptor agonists. In some embodiments, the one or more of therapeutic agents are administered after about 9 hours of administration of the one or more or 5HT2A receptor agonists. In some embodiments, the one or more of therapeutic agents are administered after about 10 hours of administration of the one or more or 5HT2A receptor agonists. In some embodiments, the one or more of therapeutic agents are administered after about 11 hours of administration of the one or more or 5HT2A receptor agonists. In some embodiments, the one or more of therapeutic agents are administered after about 12 hours of administration of the one or more or 5HT2A receptor agonists. In some embodiments, the one or more of therapeutic agents are administered after about 13 hours of administration of the one or more or 5HT2A receptor agonists. In some embodiments, the one or more of therapeutic agents are administered after about 14 hours of administration of the one or more or 5HT2A receptor agonists. In some embodiments, the one or more of therapeutic agents are administered after about 15 hours of administration of the one or more or 5HT2A receptor agonists. In some embodiments, the one or more of therapeutic agents are administered after about 16 hours of administration of the one or more or 5HT2A receptor agonists. In some embodiments, the one or more of therapeutic agents are administered after about 17 hours of administration of the one or more or 5HT2A receptor agonists. In some embodiments, the one or more of therapeutic agents are administered after about 18 hours of administration of the one or more or 5HT2A receptor agonists. In some embodiments, the one or more of therapeutic agents are administered after about 19 hours of administration of the one or more or 5HT2A receptor agonists. In some embodiments, the one or more of therapeutic agents are administered after about 20 hours of administration of the one or more or 5HT2A receptor agonists. In some embodiments, the one or more of therapeutic agents are administered after about 21 hours of administration of the one or more or 5HT2A receptor agonists. In some embodiments, the one or more of therapeutic agents are administered after about 22 hours of administration of the one or more or 5HT2A receptor agonists. In some embodiments, the one or more of therapeutic agents are administered after about 23 hours of administration of the one or more or 5HT2A receptor agonists. In some embodiments, the one or more of therapeutic agents are administered after about 24 hours of administration of the one or more or 5HT2A receptor agonists. In some embodiments, the one or more of therapeutic agents are administered after about 25 hours of administration of the one or more or 5HT2A receptor agonists. In some embodiments, the one or more of therapeutic agents are administered after about 26 hours of administration of the one or more or 5HT2A receptor agonists. In some embodiments, the one or more of therapeutic agents are administered after about 27 hours of administration of the one or more or 5HT2A receptor agonists. In some embodiments, the one or more of therapeutic agents are administered after about 28 hours of administration of the one or more or 5HT2A receptor agonists. In some embodiments, the one or more of therapeutic agents are administered after about 29 hours of administration of the one or more or 5HT2A receptor agonists. In some embodiments, the one or more of therapeutic agents are administered after about 30 hours of administration of the one or more or 5HT2A receptor agonists. In some embodiments, the one or more of therapeutic agents are administered after about 31 hours of administration of the one or more or 5HT2A receptor agonists. In some embodiments, the one or more of therapeutic agents are administered after about 32 hours of administration of the one or more or 5HT2A receptor agonists. In some embodiments, the one or more of therapeutic agents are administered after about 33 hours of administration of the one or more or 5HT2A receptor agonists. In some embodiments, the one or more of therapeutic agents are administered after about 34 hours of administration of the one or more or 5HT2A receptor agonists. In some embodiments, the one or more of therapeutic agents are administered after about 35 hours of administration of the one or more or 5HT2A receptor agonists. In some embodiments, the one or more of therapeutic agents are administered after about 36 hours of administration of the one or more or 5HT2A receptor agonists. In some embodiments, the one or more of therapeutic agents are administered after about 40 hours of administration of the one or more or 5HT2A receptor agonists. In some embodiments, the one or more of therapeutic agents are administered after about 44 hours of administration of the one or more or 5HT2A receptor agonists. In some embodiments, the one or more of therapeutic agents are administered after about 48 hours of administration of the one or more or 5HT2A receptor agonists. In some embodiments, the one or more of therapeutic agents are administered after about 52 hours of administration of the one or more or 5HT2A receptor agonists. In some embodiments, the one or more of therapeutic agents are administered after about 56 hours of administration of the one or more or 5HT2A receptor agonists. In some embodiments, the one or more of therapeutic agents are administered after about 60 hours of administration of the one or more or 5HT2A receptor agonists. In some embodiments, the one or more of therapeutic agents are administered after about 64 hours of administration of the one or more or 5HT2A receptor agonists. In some embodiments, the one or more of therapeutic agents are administered after about 68 hours of administration of the one or more or 5HT2A receptor agonists. In some embodiments, the one or more of therapeutic agents are administered after about 72 hours of administration of the one or more or 5HT2A receptor agonists.

In certain aspects, the pattern of brain activity may include hallucinogenic action in a brain. In some embodiments, the hallucinogenic action may be caused by psychedelics, e.g., serotonergics (5-HT2A receptor agonists or classical psychedelics) such as mescaline from peyote (Lophophora williamsii) or cannabinoidergics (CB-1 receptor agonists or atypical psychedelics) such as THC from cannabis (Cannabis). In some embodiments, the hallucinogenic action may be caused by dissociatives that distort perception of sight and sound and produce feelings of detachment (dissociation) from the environment, for example, antiglutamatergics (NMDA receptor antagonists or classical dissociatives) such as “laughing gas” (nitrous oxide) and ketamine, or opioidergics (x-Opioid receptor agonists or atypical dissociatives, e.g., salvinorin A from Salvia divinorum and pentazocine). In some embodiments, the hallucinogenic action may be caused by deliriants, for example, anticholinergics (muscarinic acetylcholine receptor antagonists or classical deliriants) such as tropane alkaloids such as atropine from deadly nightshade (Atropa belladonna) and diphenhydramine (Benadryl). In some embodiments, the hallucinogenic action may be caused by GABAergics (e.g., GABAA receptor agonists, some positive allosteric modulators of the GABAA receptor, hypnotics, or atypical deliriants) such as muscimol from fly agaric (Amanita muscaria) and zolpidem (Ambien).

In certain aspects, the subject is a mammal. In some embodiments, the subject is a human.

In certain aspects, the one or more 5HT2A receptor agonists include one or more serotonin receptors found in the central and peripheral nervous systems. In certain aspects, the one or more 5HT2A receptor agonists include one or more 5HT1A and/or 5HT1B and/or 5HT1D and/or 5HT1E and/or 5HT1F receptor agonists and/or antagonists. In certain aspects, the one or more 5HT2A receptor agonists include one or more 5HT2B and/or 5HT2C receptor agonists and/or antagonists. In certain aspects, the one or more 5HT2A receptor agonists include one or more 5HT2B receptor antagonists. In some embodiments, the one or more 5HT2A receptor agonists include one or more 5HT3A and/or 5HT3B and/or 5HT3C and/or 5HT3D and/or 5HT3E receptor agonists and/or receptor antagonists. In some embodiments, the one or more 5HT2A receptor agonists include one or more 5HT4 and/or 5HT5A and/or 5HT6 and/or 5HT7 receptor agonists and/or antagonists.

In one aspect, the 5HT2A receptor agonists include compounds of the following Formula (T):

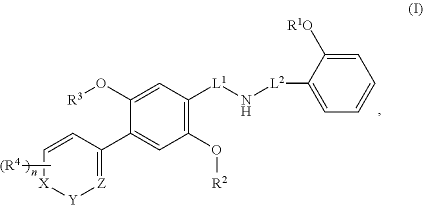

or pharmaceutically acceptable salt thereof,
wherein:
X, Y, and Z are each independently —C═ or —N═;
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl; and
n is an integer of 0 to 5.

In embodiments, in Formula (I), X is —C═, Y is —C═ and Z is —C═.

In certain embodiments, in Formula (I), X is —N═, Y is —C═ and Z is —C═.

In certain embodiments, in Formula (I), X is —N═, Y is —N═ and Z is —C═.

In certain embodiments, in Formula (I), X is —N═, Y is —C═ and Z is —N═.

In certain embodiments, in Formula (I), L¹ and L² are each independently substituted or unsubstituted C₁-C₄ alkylene.

In certain embodiments, in Formula (I), R¹, R², and R³ are each independently hydrogen, or substituted or unsubstituted C₁-C₄ alkyl.

In certain embodiments, in Formula (I), R¹ is hydrogen, and R² and R³ are each independently substituted or unsubstituted C₁-C₄ alkyl.

In certain embodiments, n is 0.

Exemplary preferred compounds of Formula (I) include the following compounds:

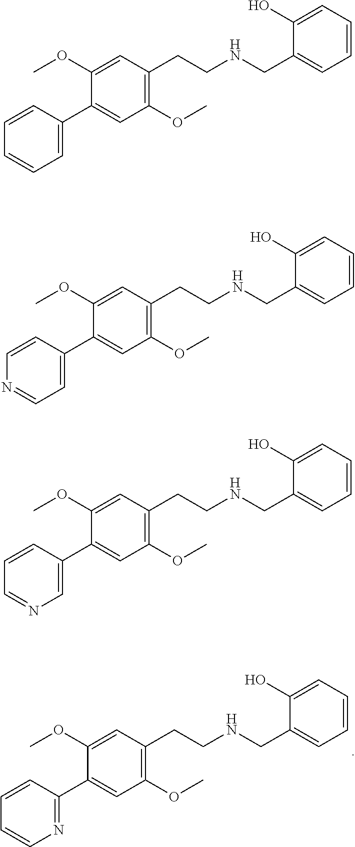 [SMILES: COc1cc(-c2ccccc2)c(OC)cc1CCNCc1ccccc1O ; COc1cc(-c2ccncc2)c(OC)cc1CCNCc1ccccc1O ; COc1cc(-c2cccnc2)c(OC)cc1CCNCc1ccccc1O ; COc1cc(-c2ccccn2)c(OC)cc1CCNCc1ccccc1O]

In one aspect, compounds of Formula (II) are provided:

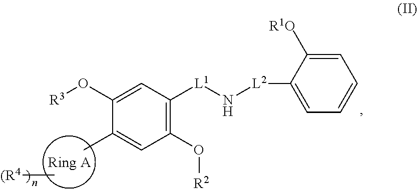

or pharmaceutically acceptable salt thereof,
wherein:
Ring A is substituted or unsubstituted 5 membered heteroaryl or heterocyclic ring containing O, S, or N;
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl; and
n is an integer of 0 to 5.

In some embodiments, in Formula(II), Ring A is

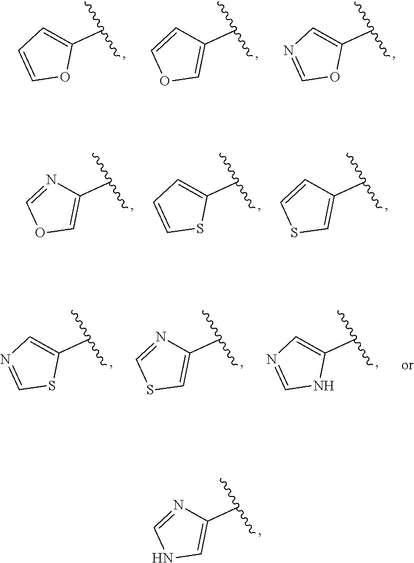

wherein the squiggly line (“ [symbol] ”) is the attachment point to the phenyl ring.

In some embodiments, in Formula (II), L¹ and L² are each independently substituted or unsubstituted C₁-C₄ alkylene.

In some embodiments, in Formula (II), R¹, R², and R³ are each independently hydrogen, or substituted or unsubstituted C₁-C₄ alkyl.

In some embodiments, in Formula (II), R¹ is hydrogen, and R² and R³ are each independently substituted or unsubstituted C₁-C₄ alkyl.

In some embodiments, in Formula (II), n is 0.

In some embodiments, in Formula (II), R² is substituted or unsubstituted C₁-C₄ alkyl.

Exemplary preferred compounds of Formula (II) include the following compounds:

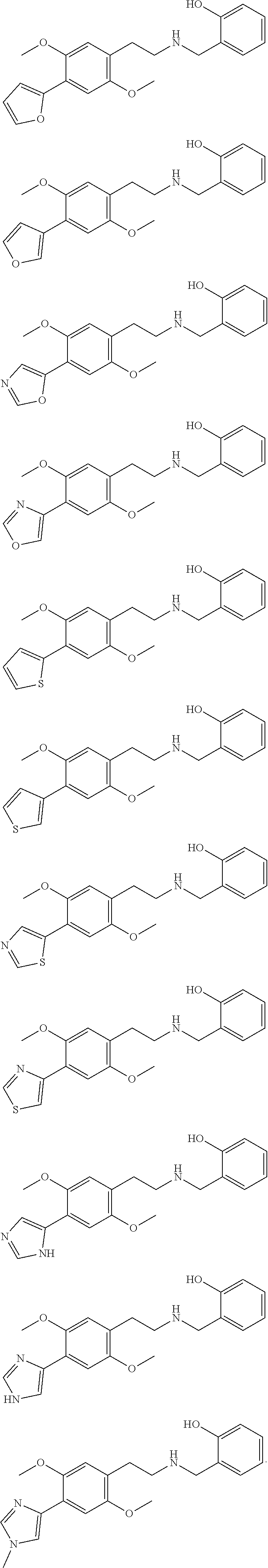

In certain aspect, compounds of Formula (III) are provided:

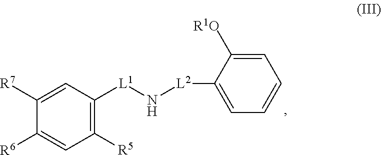

or pharmaceutically acceptable salt thereof,
wherein:
L¹ and L² are each independently or substituted or unsubstituted alkylene;
R¹ is hydrogen, or substituted or unsubstituted alkyl;
R⁵ and R⁷ are each independently hydrogen, halogen, substituted or unsubstituted alkyl, or substituted or unsubstituted heteroalkyl;
R⁶ is substituted or unsubstituted phenyl, or substituted or unsubstituted 5 membered heteroaryl or heterocyclic ring containing O, S, or N.

In some embodiments, in Formula (III), L¹ and L² are each independently substituted or unsubstituted C₁-C₄ alkylene.

In some embodiments, in Formula (III), R¹ is hydrogen, or substituted or unsubstituted C₁-C₄ alkyl.

In some embodiments, R⁵ and R⁷ are each independently hydrogen, halogen, substituted or unsubstituted C₁-C₄ alkyl, or substituted or unsubstituted 2 to 4 membered heteroalkyl. In some embodiments, R⁵ and R⁷ are each independently hydrogen, —Cl, C₁-C₄ haloalkyl, or C₁-C₄ alkoxyl.

In some embodiments, R⁶ is substituted or unsubstituted phenyl, substituted or unsubstituted pyridyl, or substituted or unsubstituted 5 membered heteroaryl or heterocyclic ring selected from

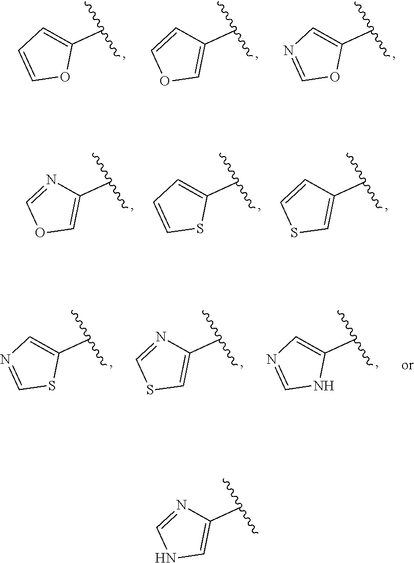

wherein the squiggly line

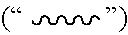

is the attachment point to the phenyl ring.

Exemplary preferred compounds of Formula (III) include the following compounds:

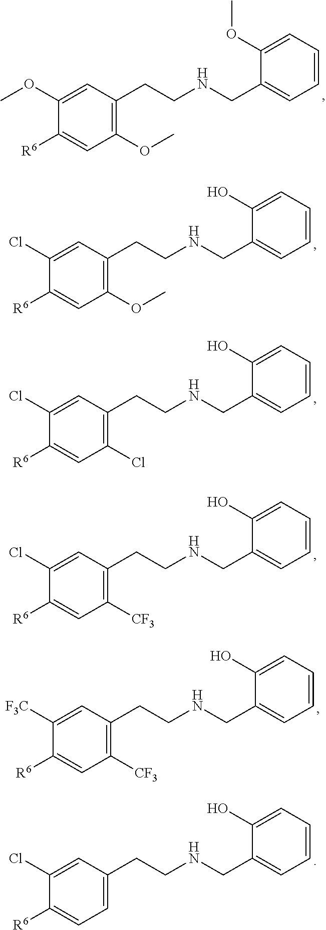

In one aspect, the 5HT2A receptor agonists include a compound having a formula (X-a),

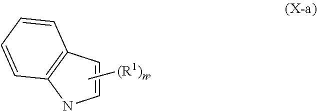

wherein
R¹ is hydrogen.
   halogen, —CN, —NR^{1A}R^{1B}, —C(O)R^{1C}, —C(O)—OR^{1C}, —C(O)NR^{1A}R^{1B}, —OR^{1D}, —NR^{1A}C (O)R^{1C}, —NR^{1A}C(O)OR^{1C}, —NR^{1A}OR^{1C}, —OR^{1D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is 1, or 2; and
R^{1A}, R^{1B}, R^{1C}, and R^{1D} are each independently hydrogen, substituted or unsubstituted alkyl, or substituted or unsubstituted heteroalkyl.

In some embodiments, R¹ is amine-substituted alkyl (e.g., CT-C3 alkyl). In certain embodiments, R¹ is —CH₂—CH₂—NH₂.

Exemplary 5HT2A receptor agonists may include, but not limited to, indolanmines having a structure of

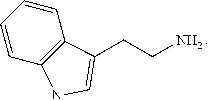 [SMILES: NCCc1c[nH]c2ccccc12]

In one aspect, the 5HT2A receptor agonists include a compound having a formula (X-b),

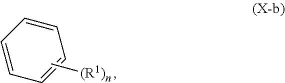

wherein n is an integer from 1 to 5. R¹ may be as described above.

In some embodiments, R¹ is amine-substituted alkyl (e.g., C₁-C₃ alkyl). In certain embodiments, R¹ is —CH₂—CH₂—NH₂.

Exemplary 5HT2A receptor agonists may include, but not limited to, indolamines having a structure of

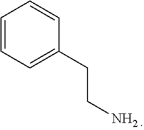

In certain aspect, the alpha7 nicotinic acetylcholine receptor modulator may include one or more selected from (+)-N-(1-azabicyclo[2.2.2]oct-3-yl)benzo[b]furan-2-carboxamide, tilorone, A-582941, AR-R17779, TC-1698, bradanicline, encenicline, GTS-21, PHA-543,613, PNU-282,987, PHA-709829, SSR-180,711, tropisetron, WAY-317,538, anabasine, acetylcholine, nicotine, epiboxidine, choline, ICH-3, PNU-120,596, NS-1738, AVL-3288, A-867744, ivermectin, nefiracetam, anandamide, α-bungarotoxin, α-conotoxin ArIB, β-caryophyllene, bupropion, dehydronorketamine, ethanol, hydroxybupropion, hydroxynorketamine, ketamine, kynurenic acid, memantine, methylcaconitine, mecamylamine, lobeline, methyllycaconitine, norketamine, and quinolizidine, dextromethorphan, amantadine, and derivatives thereof. Other examples of the alpha7 nicotinic acetylcholine receptor modulators may be used without limitation.

In certain aspect, the alpha4 nicotinic acetylcholine receptor modulators may include one or more selected from: α-conotoxin, Dextromethorphan, dihydro-β-erythroidine, mecamylamine, bupropion, 3-Bromocytisine, Acetylcholine, Cytisine, Galantamine, Epibatidine, Epiboxidine, Nicotine, A-84,543, A-366,833, ABT-418, Arecoline, Altinicline, Dianicline, Ispronicline, Pozanicline, Rivanicline, Tebanicline, TC-1827, Varenicline, Sazetidine A, N-(3-pyridinyl)-bridged bicyclic diamiines, NS-9283, Desformylflustrabromine, (−)-7-methyl-2-exo-[3′-(6-[18F]fluoropyridin-2-yl)-5′-pyridinyl]-7-azabicyclo[2.2.1]heptane, 2-fluoro-3-(4-nitro-phenyl)deschloroepibatidine, Coclaurine, Mecamylamine, α-Conotoxin, PNU-120,596, Bupropion, Dihydro-D-erythroidine, Nitrous oxide, Isoflurane, 1-(6-(((R,S)-7-Hydroxychroman-2-yl)methylamino]hexyl)-3-((S)-1-methylpyrrolidin-2-yl)pyridinium bromide, Oxantel, and derivatives thereof. Other examples of the alpha4 nicotinic acetylcholine receptor modulators may be used without limitation.

In certain aspect, the monoaminergic system modulators comprise one or more selected from 8-OH-DPAT, adatanserin, amphetamine, etoperidone, hydroxynefazodone, nefazodone, trazodone, triazoledione, vilazodone, vortioxet me, aripiprazole, asenapine, brexpiprazole, cariprazine, clozapine, lurasidone, quetiapine, zipr asidone, buspirone, eptapirone, gepirone, perospirone, tandospirone, Bay R 1531, Befiradol, BMY-14802, cannabidiol, dimemebfe, dopamine, ebalzotan, eltoprazine, enciprazine, bromocriptine, cabergoline, dihydroergotamine, ergotamine, lisuride, LSD, meth ylergometrine (methylergonovine), methysergide, pergolide, F-11461, F-12826, F-13714, F-14679, F-15063, F-15,599, flesinoxan, flibanserin, flumexadol, hypidone, lesopitron, LY-293284, LY-301317, naluzotan, NBUMP, osemozotan, oxaflozane, pardoprunox, piclozotan, rauwolscine repinotan, roxindole, RU-24,969, S-14,506, S-14671, S-15535, sarizotan, SSR-181507, Sunepitron, 5-CT, 5-MeO-DMT, 5-MT, bufotenin, DMT, indorenate, N-Me-5-HT, psilocin, psilocybin, TGBA01AD, U-92,016-A, Urapidil, Vilazodone, Xaliproden, Yohimbine, iloperidone, risperidone, sertindole, AV965, alprenolol, arteolol, cyanopindolol, iodocyanopindolol, oxprenolol, penbutolol, pindobind, pindolol, propranolol, tertatolol, BMY-7,378, CSP-2503, dotarizine, metergoline, FCE-24379, flopropione, GR-46611, lecozotan, Mefway, Metitepine (methiothepin), MIN-117 (WF-516), MPPF, NAN-190, robalzotan, S-15535, SB-649,915, SDZ 216-525, spiperone, spiramide, spiroxatrine, UH-301, WAY-100135, WAY-100635, xylamidine, acetryptine, carvedilol, ergolines (e.g., ergometrine (ergonovine)), anpirtoline, CGS-12066A, CP-93129, CP-94253, CP-122,288, CP-135807, mCPP, RU-24,969, vortioxetine, AR-A000002, carteolol, oxprenolol, penbutolol, propranolol, tertatolol, GR-127935, Isamoltane, LY-393558, SB-216641, SB-224289, SB-236057, CP-286601, L-694247, L-772405, PNU-109291, PNU-142633, alniditan, BRL-15,572, elzasonan, GR-127935, LY-310762, LY-367642, LY-456219, LY-456220, mianserin, BRL-54443, adatanserin, altanserin, cyproheptadine, hydroxyzine, ketotifen, perlapine, amda, amperozide, aripiprazole, asenapine, blonanserin, brexpiprazole, carpipramine, clocapramine, clorotepine, clozapine, fluperlapine, gevotroline, lurasidone, melperone, mosapramine, ocaperidone, olanzapine, paliperidone, quetiapine, zicronapine, zotepine, chlorprothixene, cinanserin, CSP-2503, deramciclane, dotarizine, eplivanserin, amesergide, LY-53857, LY-215,840, mesulergine, metergoline, methysergide, sergolexole, fananserin, flibanserin, glemanserin, irindalone, ketanserin, KML-010, landipirdine, medifoxamine, MIN-117 (WF-516), Naftidrofuryl, nantenine, nelotanserin, opiranserin (VVZ-149), pelanserin, phenoxybenzamine, pimavanserin, pirenperone, pizotifen, pruvansenn, rauwolscine, roluperidone, sarpogrelate, lubazodone, mepiprazole, TGBA01AD, teniloxazine, temanogrel, amoxapine, aptazapine, esmirtazapine, maprotiline, mianserin, mirtazapine, amitriptyline, chlorpromazine, fluphenazine, haloperidol, loxapine, perphenazine, pimozide, pipamperone, prochlorperazine, setoperone, spiperone, spiramide, thioridazine, thiothixene, trifluoperazine, Volinanserin, dihydroergotamine, nicergoline, 4-methylaminorex, aminorex, chlorphentermine, cloforex, dexfenfluramine, enfluramine, levofenfluramine, norfenfluramine, BW-723C86, cabergoline, dihydroergocryptine, dihydroergotamine, ergotamine, Lorcaserin, PNU-22394, Ro60-0175, Agomelatine, EGIS-7625, LY-393558, Metadoxine, PRX-08066, Ritanserin, RS-127445, SB-200646, SB-204741, SB-206553, SB-215505, SB-221284, SB-228357, SDZ SER-082, Tegaserod, and naphthylpiperazine. Other examples of the monoaminergic system modulators may be used without limitation.

In certain aspect, the cholesterol biosynthesis modulators may include one or more selected from statins, lovastatin, simvastatin, pravastatin, fluvastatin, atorvastatin, cerivastatin, and TSPO agonists comprising YL-IPA08, Ro5-4864, anthralin, alpidem, DAA-1097, DAA-1106, DPA-713, DPA-714, emapunil, etifoxine, FGIN-127, FGIN-143, GML-1. SSR-180,575, or PK-11195. Other examples of the cholesterol biosynthesis modulators may be used without limitation.

In certain aspect, the cytokines/inflammatory modulators may include one or more selected from CCL4 (MIP-1p), CCL5 (RANTES), CCL6, CCL9 (CCL10), CCL14, CCL15, CCL16, CCL23, CCL2, CCL8, CCL12, CCL16, Cenicriviroc (TAK-652, TBR-652), CCL5 (RANTES), CCL7, CCL11, CCL13, CCL15, CCL18, CCL24, CCL26, CCL28, CCL3 (MIP-1α), CCL5 (RANTES), CCL17, CCL22, Mogamulizumab, CCL3 (MIP-1α), CCL4 (MIP-1β), CCL5 (RANTES), CCL8, CCL11, CCL13, CCL14, CCL16, Aplaviroc, Cenicriviroc (TAK-652, TBR-652), INCB009471, Maraviroc, Vicriviroc, PRO-140 antibody, CCL20, CCL19, CCL21, CCL1, CCL16, CCL25, CCL27, CCL28, CCL19, CCL21, CCL25, Bertilimumab, Carlumab, CXCL6, Emoctakin, Interleukin-8 (CXCL8, GCP-1), Navarixin, Ladarixin, Reparixin (repertaxin), CXCL1 (MGSA), CXCL2, CXCL3, CXCL5, CXCL6, CXCL7, Emoctakin, Garnocestim, Interleukin-8 (CXCL8, GCP-1), Danirixin, Elubrixin, Navarixin, Ladarixin, Reparixin (repertaxin), CXCL4 (PF4), CXCL9 (MIG), CXCL10 (IP-10), CXCL11 (1-TAC), Iroplact, Eldelumab, MIF, SDF-1 (CXCL12), Ubiquitin, Mavorixafor, Plerixafor (AMD3100), Ulocuplumab, CXCL13, CXCL16, CXCL11 (I-TAC), SDF-1 (CXCL12), Plerixafor (AMD3100), Lymphotactin-α (XCL1), Lymphotactin-β (XCL2), Pateclizumab, Fractalkine (CX3CL1), CC(β) chemokines, Chemerin, Resolvin E1, ARA-290, Asialo erythropoietin, Carbamylated erythropoietin, CNTO-530, Darbepoetin alpha, Epoetin alpha, Epoetin beta, Epoetin delta, Epoetin epsilon, Epoetin gamma, Epoetin kappa, Epoetin omega, Epoetin theta, Epoetin zeta, Erythropoietin (EPO), Erythropoietin-Fc, Methoxy polyethylene glycol-epoetin beta (CERA/Mircera), Peginesatide, Pegol sihematide (EPO-018B), Filgrastim, Granulocyte colony-stimulating factor, Lenograstim, Leridistim, Lipegfilgrastim, Nartograstim, Pegfilgrastim, Pegnartograstim, Ecogramostim, Granulocyte macrophage colony-stimulating factor, Milodistim, Molgramostim, Regramostim, Sargramostim, Mavrilimumab, Namilumab, Otilimab, Cilmostim, Interleukin-34, Lanimostim, Macrophage colony-stimulating factor, Mirimostim, Agerafenib, Eltrombopag, Pegacaristim, Promegapoietin, Romiplostim, Thrombopoietin (THPO, MGDF), Albinterferon, Interferon alpha (interferon alpha, IFN-α), Interferon alpha (IFNA1, IFNA2, IFNA4, IFNA5, IFNA6, IFNA7, IFNA8, IFNA10, IFNA13, IFNA14, IFNA16, IFNA17, IFNA21), Interferon alpha 2a, Interferon alpha 2b, Interferon alpha n1, Interferon alphacon-1, Interferon alpha-n3, Interferon beta (IFN-β) (IFNB1, IFNB3), Interferon beta 1a, Interferon beta 1b, Interferon kappa (IFN-ε/κ/τ/ζ, IFNK), Interferon omega (IFN-ω, IFNW1), Peginterferon alpha-2a, Peginterferon alpha-2b, Anifrolumab, Faralimomab, MEDI-545, Rontalizumab, Sifalimumab, Bifarcept, Interferon gamma (IFN-γ), Interferon gamma 1b, Emapalumab, Fontolizumab, Interleukin 1 (α, β), Mobenakin, Pifonakin, AF-12198, Anakinra, IL-1RA, Isunakinra, Canakinumab, Gevokizumab, Lutikizumab, Decoy receptors: Rilonacept (IL-1 Trap), Adargileukin alpha, Aldesleukin, Celmoleukin, Denileukin diftitox, Interleukin 2, Pegaldesleukin, Teceleukin, Tucotuzumab celmoleukin, Basiliximab, Daclizumab (dacliximab), Inolimomab, Daniplestim, Interleukin 3, Leridistim, Milodistim, Muplestim, Promegapoietin, Binetrakin, Interleukin 4, Interleukin 13, Pitrakinra, Dupilumab, Pascolizumab, Interleukin 5, YM-90709, Benralizumab, Mepolizumab, Reslizumab, TPI ASM8, Atexakin alpha, Interleukin 6, ARGX-109, Clazakizumab, Elsilimomab, mAb 1339, Olokizumab, Sarilumab, Siltuximab, Sirukumab, Tocilizumab, Levilimab: Interleukin 7, Interleukin 9, Enokizumab, Ilodecakin, Interleukin 10 (CSIF), Interleukin 11 (AGIF), Oprelvekin, Edodekin alpha, Interleukin 12, Briakinumab, Ustekinumab, Binetrakin, Cintredekin besudotox, Interleukin 4, Interleukin 13, Anrukinzumab, Lebrikizumab, Tralokinumab, ALT-803, Interleukin 15, Interleukin 17 (A, B, C, D, E (interleukin 25), Brodalumab, Ixekizumab, Perakizumab, Remtolumab, Secukinumab, Vunakizumab, Iboctadekin, Interleukin 18, Interleukin 37, Tadekinig, IL18BP, Interleukin 19, Interleukin 20, Interleukin 24, Fletikumab, Denenicokin, Interleukin 21, NNC0114-0005, NNC0114-0006, Interleukin 22, Fezakinumab, Interleukin 23 (SGRF), Brazikumab, Briakinumab, Guselkumab, Risankizumab, Tildrakizumab, Ustekinumab, Interleukin 27 (interleukin 30), Interferon λ4 (IFN-λ4), Interleukin 28 (A (IFN-λ2), B (IFN-λ3)), Interleukin-29 (IFN-λ1), Interleukin 31, Interleukin 33, Interleukin 36 (α, β, γ), Interleukin 38, IL-36RA, Interleukin 14 (taxilin alpha, HMW-BCGF), Interleukin 16, Interleukin 24, Interleukin 26, Interleukin 32, Interleukin 34, Interleukin 35, Efavaleukin alpha, Efineptakin alpha, Activin (A, B, AB), Avotermin, BMP (10), Cetermin, GDF (2 (BMP9)), TGFβ (1, 2, 3): DMH-1, DMH-2, Dorsomorphin (BML-275), K-02288, ML-347 (LDN-193719, VU0469381), Ascrinvacumab, K-02288, ML-347 (LDN-193719, VU0469381), Dalantercept, Disitertide, Activin (A, B, AB), AMH (MIS), Avotermin, BMP (5, 6, 7, 8A, 8B), Eptotermin alpha, TGFβ (1, 2, 3), AMH (MIS), BMP (2, 4, 5, 6, 7, 8A, 8B), Dibotermin alpha, Eptotermin alpha, DMH-2, Dorsomorphin (BML-275), K-02288, Activin (A, B, AB), GDF (1, 3, 11 (BMP11)), Myostatin (GDF8), Nodal, Inhibin (A, B), Lefty (1, 2), A 83-01, SB-431542, SB-505124, Avotermin, GDF (10 (BMP3B), 11 (BMP11)), TGFβ (1, 2, 3), Fresolimumab, Lerdelimumab, Metelimumab, A 83-01, D-4476, GW-788388, LY-364947, LY-2109761, Galunisertib (LY-2157299), R-268712, RepSox (E-616452, SJN-2511), SB-431542, SB-505124, SB-525334, SD-208, BMP (2, 4, 5, 6, 7, 8A, 8B, 15, GDF9B)), BAMBI, Cerberus (CER1), Chordin, DAN (PARN), Decorin, Follistatin, Gremlin (Drm), LTBP1, Noggin, TGIF, Thrombospondin 1 (THBS1), Tomoregulin 1, Stamulumab, TRC105, Endoglin, Lymphotoxin, Baminercept, Plusonermin, Sonermin, Tasonfermin, Afelimomab, Certolizumab pegol, Golimumab, Infliximab, Nerelimomab, Ozoralizumab, Remtolumab, Placulumab, Belimumab, Brentuximab vedotin, Conatumumab, Dacetuzumab, Denosumab, Drozitumab, Enavatuzumab, Iratumumab, Lexatumumab, Lucatumumab, Mapatumumab, Oxelumab, Ruplizumab, Tabalumab, Tavolixizumab, Teneliximab, Tigatuzumab, Toralizumab, Urelumab, Utomilumab, Varlilumab, Vorsetuzumab, Vorsetuzumab mafodotin, Abrocitinib, Baricitinib. Filgotinib. Momelotinib, Oclacitinib, Peficitinib, Ruxolitinib, Tofacitinib (tasocitinib, CP-690550), Upadacitinib, Atiprimod, AZD-1480, Baricitinib, CHZ868, Cucurbitacin I (elatericin B, JSI-124), CYT387, Lestaurtinib, NSC-7908, NSC-33994, Pacritinib, Peficitinib, Ruxolitinib, SD-1008, Tofacitinib (tasocitinib, CP-690550), Cercosporamiide, Decernotinib (VX-509), Peficitinib, TCS-21311, Tofacitinib (tasocitinib, CP-690550), WHI-P 154, ZM-39923, ZM-449829, Cardiotrophin 1 (CT-1), FMS-like tyrosine kinase 3 ligand (FLT3L), Leukemia/leukocyte inhibitory factor (LIF), Oncostatin M (OSM), Thymic stromal lymphopoietin (TSLP), lestaurtinib, midostaurin, quizartinib, sorafenib, and sunitinib. Other examples of the cytokines/inflammatory modulators may be used without limitation.

In certain aspects, the trace amine-associated receptor modulators comprise one or more selected from SEP-363856, RO5166017, RO5256390, RO5203648, RO5263397, RO5073012, and o-PIT.

In certain embodiments, (i) one or more or 5HT2A (5-hydroxy-tryptanine) receptor agonists to the subject, and (ii) one or more of therapeutic agents are administered by a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal).

### III. Compositions

In an aspect, provided are compositions (e.g., pharmaceutical composition) for use of treating or ameliorating altered patterns of brain activity in a subject. The compositions may include (i) one or more or 5HT2A (5-hydroxy-tryptamine) receptor agonists to the subject, and (ii) one or more of therapeutic modulatory agents. In some embodiments, the one or more of therapeutic agents include (a) alpha7 nicotinic acetylcholine receptor modulators; (b) alpha4 nicotinic acetylcholine receptor modulators; (c) monoaminergic system modulators; (d) cholesterol biosynthesis modulators; (e) cytokines/inflammatory modulators; (f) statin compounds; (g) immune system modulators and/or (h) trace amine-associated receptor modulators.

The pharmaceutical composition may be suitably formulated for co-administration. The “co-administer” is meant that a composition described herein is administered at the same time, just prior to, or just after the administration of one or more additional therapies. The compounds provided herein can be administered alone or can be coadministered to the patient.

Coadministration is meant to include simultaneous or sequential administration of the compounds individually or in combination (more than one compound). Thus, the preparations can also be combined, when desired, with other active substances (e.g. to reduce metabolic inhibition).

In certain aspects, the one or more of therapeutic agents are contained in a composition mixed or combined with the one or more or 5HT2A (5-hydroxy-tryptamine) receptor agonists. In certain aspects, the one or more of therapeutic agents and the one or more or 5HT2A (5-hydroxy-tryptamine) receptor agonists are contained in separate compositions.

The pharmaceutical composition may suitably include pharmaceutically acceptable carriers or excipients.

In certain embodiments, the pharmaceutical composition is formulated for intranasal or inhalation, like aerosols, inhalers, nebulizers and vaporizers, administration. In certain embodiments, the pharmaceutical composition is formulated for intraoral administration. In certain embodiments, the pharmaceutical composition is formulated for parenteral administration, such as intravenous, intramuscular, intradermal, subcutaneous, intraosseous, caudal, intrathecal or intraperitoneal administration. In certain embodiments, the pharmaceutical composition is formulated for administration by infusion. In certain embodiments, the pharmaceutical composition is formulated for sublingual, orally disintegrating or buccal administration. In certain embodiments, the pharmaceutical composition is formulated for ophthalmic or otic administration.

In certain embodiments of the present invention, the pharmaceutical composition (e.g., a pharmaceutical composition formulated for, for example, oral (e.g., intraoral administration or a tablet, caplet, gelcap, cap or capsule composition), rectal, vaginal, intransal, inhalation, otic, ophthalmic, topical, sublingual, orally disintegration, buccal, parenteral, intravenous, subcutaneous or intramuscular administration, or formulated for administration by infusion, includes an effective amount of respectively (i) one or more or 5HT2A (5-hydroxy-tryptamine) receptor agonists to the subject, and (ii) one or more of therapeutic agents.

### IV. Kits

In an aspect provided are kits comprising the compositions as described above.

In certain aspect, the kit includes compositions (e.g., pharmaceutical compositions) including (i) one or more or 5HT2A(5-hydroxy-tryptamine) receptor agonists to the subject, and (ii) one or more of therapeutic agents. In certain aspect, the one or more therapeutic agents include one or more selected from (a) alpha7 nicotinic acetylcholine receptor modulators; (b) alpha4 nicotinic acetylcholine receptor modulators; (c) monoaminergic system modulators; (d) cholesterol biosynthesis modulators; (e) cytokines/inflammatory modulators; (f) statin compounds; (g) immune system modulators and/or (h) trace amine-associated receptor modulators.

In certain aspects, the one or more of therapeutic agents and the one or more or 5HT2A (5-hydroxy-tryptamine) receptor agonists are contained in a composition as mixed or combined each other. In certain aspects, the one or more of therapeutic agents and the one or more or 5HT2A (5-hydroxy-tryptamine) receptor agonists are in separate compositions.

In certain aspects, the compounds (e.g., modulators) suitably can be packaged in suitable containers labeled, for example, for use as described herein. In addition, an article of manufacture or kit further may include, for example, packaging materials, instructions for use, delivery devices, for treating or monitoring the specified condition. The kit may also include a legend (e.g., a printed label or insert or other medium describing the product's use). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions for example can be provided in a concentrated form with a diluent and instructions for dilution.

The subject (for example, human patient exhibiting treatment resistant depression) may receive a wide range of dosage of a 5HT2A receptor agonist, for example a 5HT2A receptor agonist may be suitably administered in amounts of 0.1, 0.2, 0.0.3, 0.5 or 1 mg/kg of patient bodyweight daily or weekly.

The subject (for example, human patient exhibiting treatment resistant depression) also may receive a wide range of dosage of a 5HT2A receptor modulator compound in combination with the 5HT2A receptor agonist. For example, a 5HT2A receptor modulator compound may be administered at 0.1, 0.2, 0.3, 0.5 or 1 mg/kg of patient bodyweight daily or weekly.

### EXAMPLE

### Example 1: Treatment Protocol for a Subject Having Treatment Resistant Depression

A human patient is selected for treatment exhibiting continued depressive symptoms after 3-month treatment with Zoloft.

The subject receives 0.2 mg of psilocin daily followed by 10 mg of lovastatin every 24 or 36 hours.

### Example 2: Synthesis

The present compounds can be readily including for example by reaction of a primary amine with a substituted alkyl halide. For instance, cesium hydroxide can be used in a coupling reaction which can promote a selective N-monoalkylation of primary amines to prepare various secondary amines efficiently.

Compounds (1A-1 to 2A-2) are synthesized according to following Scheme 1.

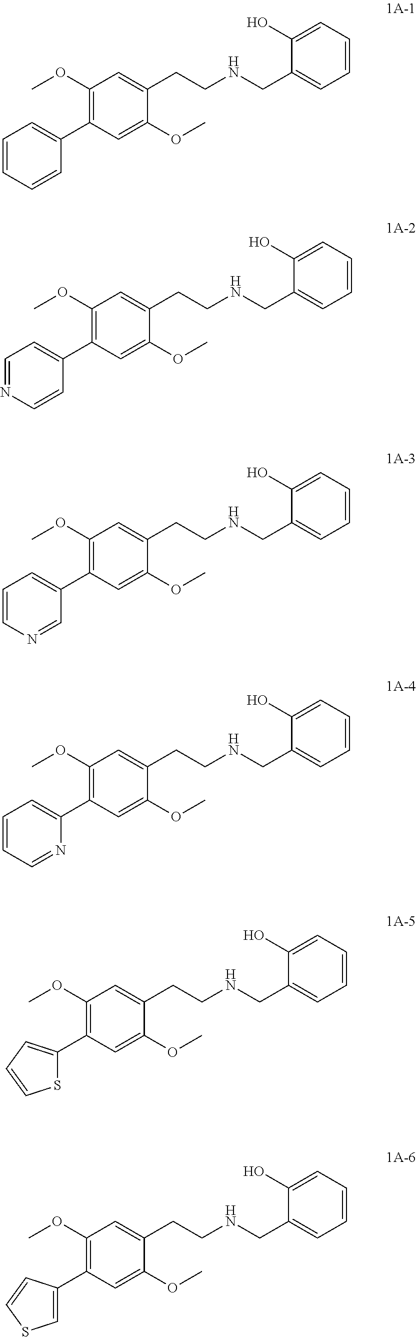 [SMILES: COc1cc(-c2ccccc2)c(OC)cc1CCNCc1ccccc1O ; COc1cc(-c2ccncc2)c(OC)cc1CCNCc1ccccc1O ; COc1cc(-c2cccnc2)c(OC)cc1CCNCc1ccccc1O ; COc1cc(-c2ccccn2)c(OC)cc1CCNCc1ccccc1O ; COc1cc(-c2cccs2)c(OC)cc1CCNCc1ccccc1O ; COc1cc(-c2ccsc2)c(OC)cc1CCNCc1ccccc1O]

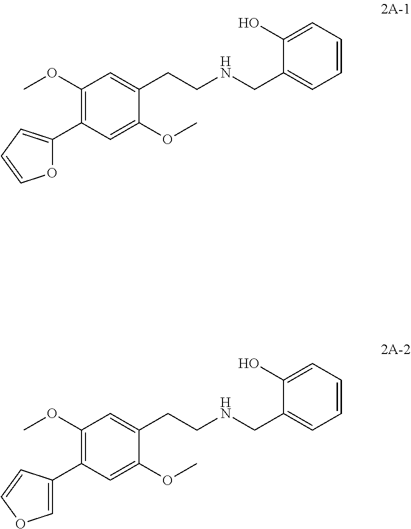 [SMILES: COc1cc(-c2ccco2)c(OC)cc1CCNCc1ccccc1O ; COc1cc(-c2ccoc2)c(OC)cc1CCNCc1ccccc1O]

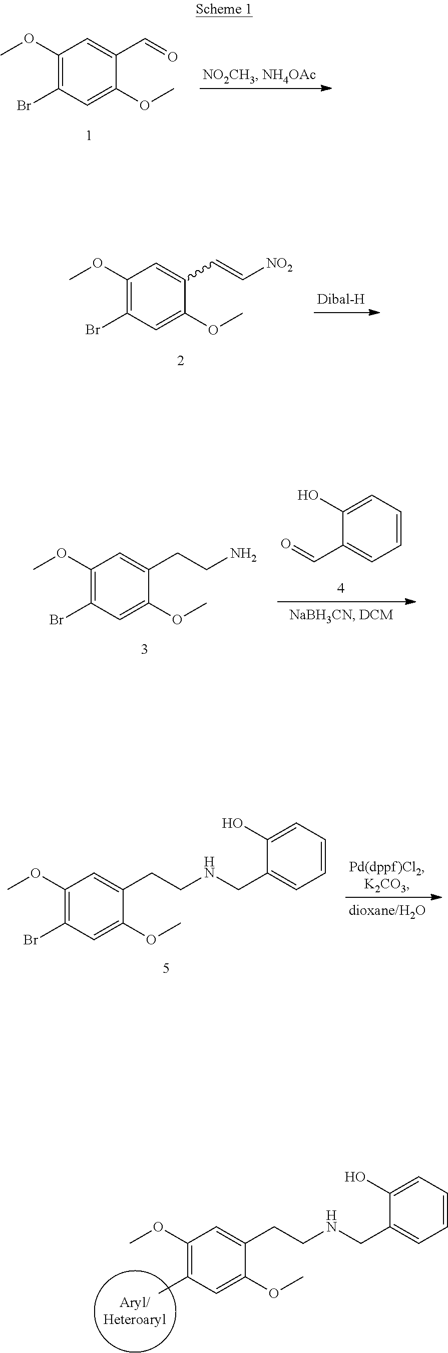

### Step 1

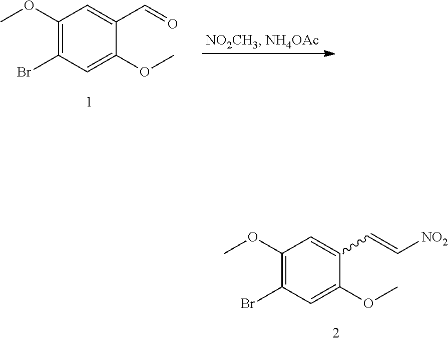

To a solution of 4-bromo-2,5-dimethoxy benzaldehyde (1, 5.0 g, 20.4 mmol) in MeNO₂ (50 mL), was added NH₄OAc (1.58 g, 20.4 mmol) and the reaction mixture was stirred at reflux for 1.5 h. After cooling down, excess MeNO₂ was removed in vacuo and the residue was dissolved in EA (100 mL). The organic layer was washed with water, dried over MgSO₄, filtered and evaporated in vacuo. The residue was purified by flash chromatography (PE/EA=9:1) to give the title compound 1-bromo-2,5-dimethoxy-4-(2-nitrovinyl)benzene (2, 4.2 g, purity:85%) as a yellow solid.

### Step 2

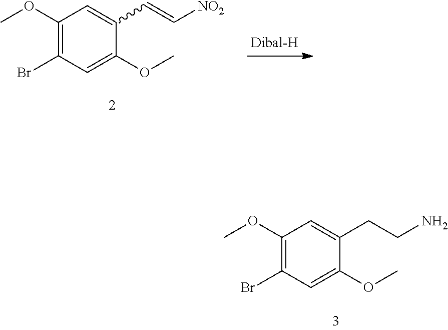 [SMILES: COc1cc(C=C[N+](=O)[O-])c(OC)cc1Br ; COc1cc(CCN)c(OC)cc1Br]

To a solution of 1-bromo-2,5-dimethoxy-4-(2-nitrovinyl)benzene (2) (4.2 g, 14.58 mmol) in THF (20 mL) was dropwise a solution of Dibal-H (102 mL, 102 mmol, 1M in THF) at 0° C., then the mixture was heated to 60° C. for 2 hours. After cooling down, NH₄Cl solution added, extracted with EA, dried over MgSO₄, filtered and concentrated to get the crude product 2-(4-bromo-2,5-dimethoxyphenyl)ethanamine (3, crude), which was used directly at next step without further purification.

### Step 3

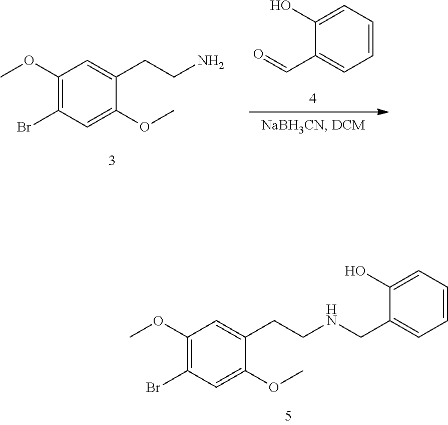 [SMILES: COc1cc(CCN)c(OC)cc1Br ; O=Cc1ccccc1O ; COc1cc(CCNCc2ccccc2O)c(OC)cc1Br]

A mixture of 2-(4-bromo-2,5-dimethoxyphenyl)ethanamine (3, crude) and 2-hydroxybenzaldehyde (4, 1.5 g, 12.4 mmol) in DCM (40 mL) was added NaBH₃CN (1.6 g, 24.5 mmol) in portions and one drop AcOH, then the mixture was stirred overnight at rt. NaHCO₃ solution added, extracted with DCM, dried over MgSO₄, filtered and evaporated in vacuo. The residue was purified by flash chromatography (PE/EA=2:1) to give the title compound 2-(((4-bromo-2,5-dimethoxyphenethyl)amino)methyl)phenol (5, 1.6 g, yield: 32%) as a colorless oil.

### Step 4

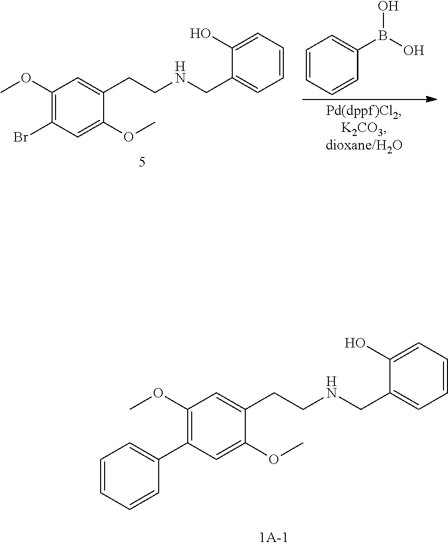

A mixture of 2-(((4-bromo-2,5-dimethoxyphenethyl)amino)methyl)phenol (5, 400 mg, 1.0 mmol), phenylboronic acid (200 mg, 1.64 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((2-(2,5-dimethoxy-[1,1′-biphenyl]-4-yl)ethyl)amino)methyl)phenol (1A-1, 56 mg, yield: 15%) as a brown solid.

### Step 5

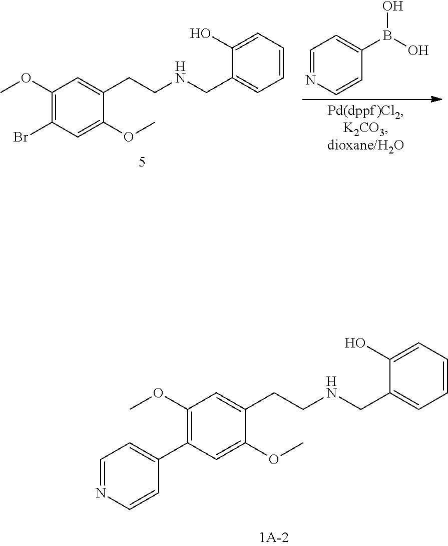

A mixture of 2-(((4-bromo-2,5-dimethoxyphenethyl)amino)methyl)phenol (5, 400 mg, 1.0 mmol), pyridin-4-ylboronic acid (200 mg, 1.64 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((2,5-dimethoxy-4-(pyridin-4-yl)phenethyl)amino)methyl)phenol (1A-2, 120 mg, yield: 26%) as a yellow solid.

### Step 6

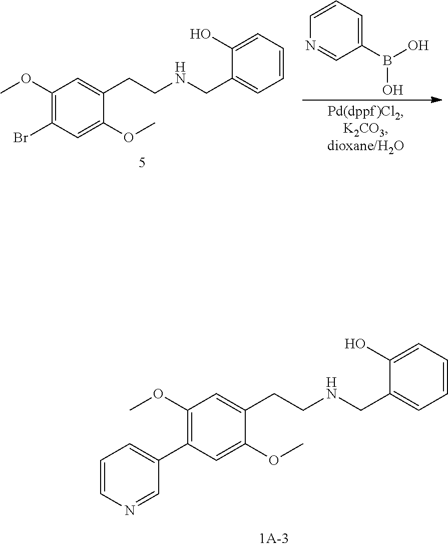

A mixture of 2-(((4-bromo-2,5-dimethoxyphenethyl)amino)methyl)phenol (5, 400 mg, 1.0 mmol), pyridin-3-ylboronic acid (200 mg, 1.64 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((2,5-dimethoxy-4-(pyridin-3-yl)phenethyl)amino)methyl)phenol (1A-3, 70 mg, yield: 15%) as a yellow solid.

### Step 7

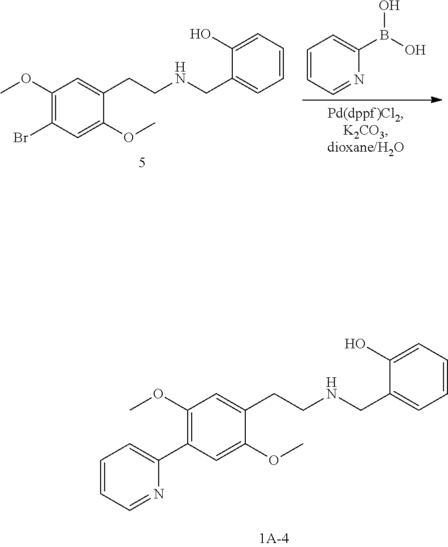

A mixture of 2-(((4-bromo-2,5-dimethoxyphenethyl)amino)methyl)phenol (5, 500 mg, 1.3 mmol), pyridin-2-ylboronic acid (400 mg, 3.2 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((2,5-dimethoxy-4-(pyridin-2-yl)phenethyl)amino)methyl)phenol (1A-4, 110 mg, yield: 18%) as a yellow solid.

### Step 7

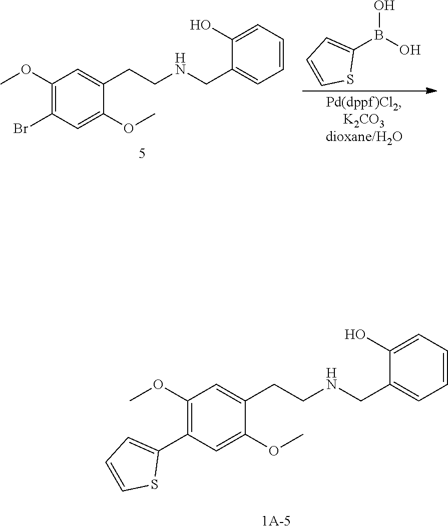

A mixture of 2-(((4-bromo-2,5-dimethoxyphenethyl)amino)methyl)phenol (5, 400 mg, 1.0 mmol), thiophen-2-ylboronic acid (300 mg, 1.6 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((2,5-dimethoxy-4-(thiophen-2-yl)phenethyl)amino)methyl)phenol (1A-5, 120 mg, yield: 26%) as a white solid.

### Step 8

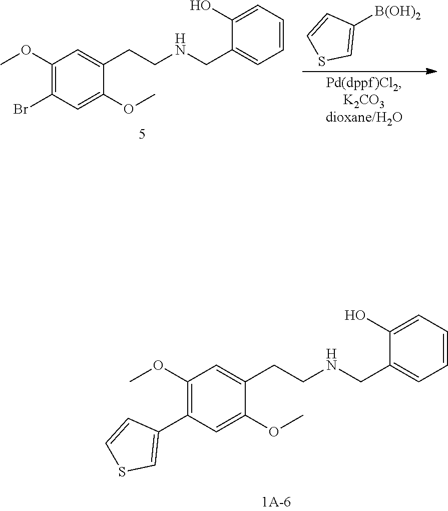

A mixture of 2-(((4-bromo-2,5-dimethoxyphenethyl)amino)methyl)phenol (5, 400 mg, 1.0 mmol), thiophen-3-ylboronic acid (300 mg, 1.6 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((2,5-dimethoxy-4-(thiophen-3-yl)phenethyl)amino)methyl)phenol (1A-6, 110 mg, yield: 25%) as a white solid

### Step 9

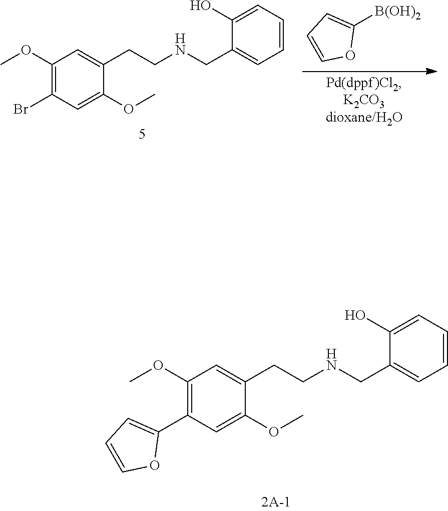

A mixture of 2-(((4-bromo-2,5-dimethoxyphenethyl)amino)methyl)phenol (5, 400 mg, 1.0 mmol), furan-2-ylboronic acid (180 mg, 1.6 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((4-(furan-2-yl)-2,5-dimethoxyphenethyl)amino)methyl)phenol (2A-1, 120 mg, yield: 24%) as a white solid.

### Step 10

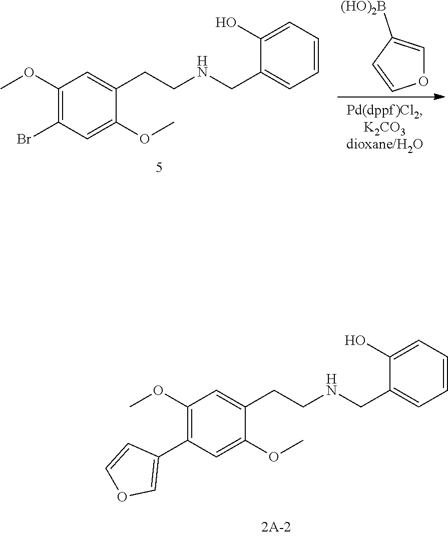 [SMILES: COc1cc(CCNCc2ccccc2O)c(OC)cc1Br ; OB(O)c1ccoc1 ; COc1cc(-c2ccoc2)c(OC)cc1CCNCc1ccccc1O]

A mixture of 2-(((4-bromo-2,5-dimethoxyphenethyl)amino)methyl)phenol (5, 400 mg, 1.0 mmol), furan-3-ylboronic acid (180 mg, 1.6 mmol), K₂CO₃ (415 mg, 3.0 mmol) and Pd(dppf)Cl₂ (75 mg, 0.1 mmol) in dioxane/H₂O (20 mL/2.0 mL) was heated to reflux overnight. After cooling down, NH₄Cl solution added, extracted with EA, dried over MgSO₄, filtered and evaporated in vacuo. The residue was purified by flash chromatography (PE/EA=2:1) and Prep-HPLC to give the tile compound 2-(((4-(furan-2-yl)-2,5-dimethoxyphenethyl)amino)methyl)phenol (2A-2, 58 mg, yield: 16%) as a brown solid.

1. A method of treating or ameliorating a 5HT2A mediated condition in a subject, the method comprising administering:
(i) one or more or 5HT2A receptor agonists, and
(ii) one or more 5HTA receptor modulator compounds.

2. The method of claim 1 wherein the 5HTA receptor modulator compounds are selected from:
a) alpha7 nicotinic acetylcholine receptor modulators;
b) alpha4 nicotinic acetylcholine receptor modulators;
c) monoaminergic system modulators;
d) cholesterol biosynthesis modulators;
e) cytokines/inflammatory modulators;
(f) statin compounds;
(g) immune system modulators; and/or
(h) trace amine-associated receptor modulators

3-6. (canceled)

7. The method of claim 1 wherein the one or more 5HT2A receptor modulator compounds is LSD ((lysergic acid diethylamide), psilocin, mescaline, (±)-2,5-dimethoxy-4-iodoamphetamine hydrochloride (DOI), indoleamine and/or phenethylamine

8. The method of claim 1, wherein the 5HT2A receptor agonists comprises a compound having a formula (I): 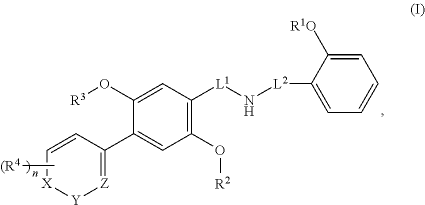
or pharmaceutically acceptable salt thereof,
wherein:
X, Y, and Z are each independently —C═ or —N═;
L¹ and L² are each independently a bond or substituted or unsubstituted alkylene;
R¹, R², and R³ are each independently hydrogen, or substituted or unsubstituted alkyl;
R⁴ is each independently halogen, or substituted or unsubstituted alkyl; and
n is an integer of 0 to 5.

9-13. (canceled)

14. The method of claim 8, wherein compound of the following Formula (III): 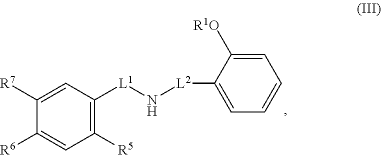
or pharmaceutically acceptable salt thereof,
wherein:
L¹ and L² are each independently a bond or substituted or unsubstituted alkylene;
R¹ is hydrogen, or substituted or unsubstituted alkyl;
R⁵ and R⁷ are each independently hydrogen, halogen, substituted or unsubstituted alkyl, or substituted or unsubstituted heteroalkyl;
R⁶ is substituted or unsubstituted phenyl, or substituted or unsubstituted 5 membered heteroaryl or heterocyclic ring containing O, S, or N.

15. (canceled)

16. The method of claim 1 wherein the 5HT2A receptor agonists comprises a compound having a formula (X-a), 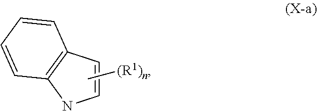
wherein
R¹ is hydrogen,
halogen, —CN, —NR^{1A}R^{1B}, —C(O)R^{1C}, —C(O)—OR^{1C}, —C(O)NR^{1A}R^{1B}, —OR^{1D}, —NR^{1A}C(O) R^{1C}, —NR^{1A}C(O)OR^{1C}, —NR^{1A}OR^{1C}, —OR^{1D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is 1, or 2; and
R^{1A}, R^{1B}, R^{1C}, and R^{1D} are each independently hydrogen, substituted or unsubstituted alkyl, or substituted or unsubstituted heteroalkyl.

17. The method of claim 1, wherein the 5HT2A receptor agonists comprises a compound having a formula (X-b), 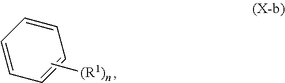
wherein
R¹ is hydrogen,
halogen, —CN, —NR^{1A}R^{1B}, —C(O)R^{1C}, —C(O)—OR^{1C}, —C(O)NR^{1A}R^{1B}, —OR^{1D}, —NR^{1A}C(O) R^{1C}, —NR^{1A}C(O)OR^{1C}, —NR^{1A}OR^{1C}, —OR^{1D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer from 1 to 5; and
R^{1A}, R^{1B}, R^{1C}, and R^{1D} are each independently hydrogen, substituted or unsubstituted alkyl, or substituted or unsubstituted heteroalkyl.

18-19. (canceled)

20. The method of claim 2 wherein the alpha7 nicotinic acetylcholine receptor modulator is selected from: (+)-N-(1-azabicyclo[2.2.2]oct-3-yl)benzo[b]furan-2-carboxamide, tilorone, A-582941, AR-R17779, TC-1698, bradanicline, encenicline, GTS-21, PHA-543,613, PNU-282,987, PHA-709829, SSR-180,711, tropisetron, WAY-317,538, anabasine, acetylcholine, nicotine, epiboxidine, choline, ICH-3, PNU-120,596, NS-1738, AVL-3288, A-867744, ivermectin, nefiracetam, anandamide, α-bungarotoxin, α-conotoxin ArIB, β-caryophyllene, bupropion, dehydronorketamine, ethanol, hydroxybupropion, hydroxynorketamine, ketamine, kynurenic acid, memantine, methylcaconitine, mecamylamine, lobeline, methyllycaconitine, norketamine, and quinolizidine, dextromethorphan, amantadine, and derivatives thereof.

21. (canceled)

22. The method of claim 2 wherein the monoaminergic system modulators comprise one or more selected from 8-OH-DPAT, Adatanserin, Amphetamine,
etoperidone, hydroxynefazodone, nefazodone, trazodone, triazoledione, vilazodone, vortioxetine, aripiprazole, asenapine, brexpiprazole, cariprazine, clozapine, lurasidone, quetiapine, ziprasidone, buspirone, eptapirone, gepirone, perospirone, tandospirone, Bay R 1531, Befiradol, BMY-14802, Cannabidiol, Dimemebfe, Dopamine, Ebalzotan, Eltoprazine, Enciprazine, bromocriptine, cabergoline, dihydroergotamine, ergotamine, lisuride, LSD, methylergometrine (methylergonovine), methysergide, pergolide, F-11461, F-12826, F-13714, F-14679, F-15063, F-15,599, Flesinoxan, Flibanserin, Flumexadol, Hypidone, Lesopitron, LY-293284, LY-301317, Naluzotan, NBUMP, Osemozotan, Oxaflozane, Pardoprunox, Piclozotan, Rauwolscine Repinotan, Roxindole, RU-24,969, S-14,506, S-14671, S-15535, Sarizotan, Serotonin (5-HT), SSR-181507, Sunepitron, 5-CT, 5-MeO-DMT, 5-MT, bufotenin, DMT, indorenate, N-Me-5-HT, psilocin, psilocybin, TGBA01AD, U-92,016-A, Urapidil, Vilazodone, Xaliproden, Yohimbine, iloperidone, risperidone, sertindole, AV965, alprenolol, arteolol, cyanopindolol, iodocyanopindolol, oxprenolol, penbutolol, pindobind, pindolol, propranolol, tertatolol, BMY-7,378, CSP-2503, Dotarizine, metergoline, FCE-24379, Flopropione, GR-46611, Lecozotan, Mefway, Metitepine (methiothepin), MIN-117 (WF-516), MPPF, NAN-190, Robalzotan, S-15535, SB-649,915, SDZ 216-525, Spiperone, Spiramide, Spiroxatrine, UH-301, WAY-100135, WAY-100635, Xylamidine, Acetryptine, Carvedilol, Ergolines (e.g., ergometrine (ergonovine)), Anpirtoline, CGS-12066A, CP-93129, CP-94253, CP-122,288, CP-135807, mCPP, RU-24,969, Vortioxetine, AR-A000002, carteolol, oxprenolol, penbutolol, propranolol, tertatolol, GR-127935, Isamoltane, LY-393558, SB-216641, SB-224289, SB-236057, CP-286601, L-694247, L-772405, PNU-109291, PNU-142633, Alniditan, BRL-15,572, Elzasonan, GR-127935, LY-310762, LY-367642, LY-456219, LY-456220, Mianserin, BRL-54443, Adatanserin, Altanserin, cyproheptadine, hydroxyzine, ketotifen, perlapine, AMDA, amperozide, aripiprazole, asenapine, blonanserin, brexpiprazole, carpipramine, clocapramine, clorotepine, clozapine, fluperlapine, gevotroline, lurasidone, melperone, mosapramine, ocaperidone, olanzapine, paliperidone, quetiapine, zicronapine, zotepine, Chlorprothixene, Cinanserin, CSP-2503, Deramciclane, Dotarizine, Eplivanserin, amesergide, LY-53857, LY-215,840, mesulergine, metergoline, methysergide, sergolexole, Fananserin, Flibanserin, Glemanserin, Irindalone, Ketanserin, KML-010, Landipirdine, Medifoxamine, MIN-117 (WF-516), Naftidrofuryl, Nantenine, Nelotanserin, Opiranserin (VVZ-149), Pelanserin, Phenoxybenzamine, Pimavanserin, Pirenperone, Pizotifen, Pruvanserin, Rauwolscine, Roluperidone, Sarpogrelate, lubazodone, mepiprazole, TGBA01AD, Teniloxazine, Temanogrel, amoxapine, aptazapine, esmirtazapine, maprotiline, mianserin, mirtazapine, amitriptyline, chlorpromazine, fluphenazine, haloperidol, loxapine, perphenazine, pimozide, pipamperone, prochlorperazine, setoperone, spiperone, spiramide, thioridazine, thiothixene, trifluoperazine, Volinanserin, dihydroergotamine, nicergoline, 4-Methylaminorex, Aminorex, chlorphentermine, cloforex, dexfenfluramine, enfluramine, levofenfluramine, norfenfluramine, BW-723C86, cabergoline, dihydroergocryptine, dihydroergotamine, ergotamine, Lorcaserin, PNU-22394, Ro60-0175, Agomelatine, EGIS-7625, LY-393558, Metadoxine, PRX-08066, Ritanserin, RS-127445, SB-200646, SB-204741, SB-206553, SB-215505, SB-221284, SB-228357, SDZ SER-082, Tegaserod, and naphthylpiperazine.

23. The method of claim 2 wherein the cholesterol biosynthesis modulators comprise one or more selected from statins, lovastatin, simvastatin, pravastatin, fluvastatin, atorvastatin, cerivastatin, and TSPO agonists comprising YL-IPA08, Ro5-4864, Anthralin, Alpidem, DAA-1097, DAA-1106, DPA-713, DPA-714, Emapunil, Etifoxine, FGIN-127, FGIN-143, GML-1, SSR-180,575, or PK-11195.

24. The method of claim 2 wherein the cytokines/inflammatory modulators comprise one or more selected from CCL4 (MIP-1β), CCL5 (RANTES), CCL6, CCL9 (CCL10), CCL14, CCL15, CCL16, CCL23, CCL2, CCL8, CCL12, CCL16, Cenicriviroc (TAK-652, TBR-652), CCL5 (RANTES), CCL7, CCL11, CCL13, CCL15, CCL18, CCL24, CCL26, CCL28, CCL3 (MIP-1α), CCL5 (RANTES), CCL17, CCL22, Mogamulizumab, CCL3 (MIP-1α), CCL4 (MIP-1β), CCL5 (RANTES), CCL8, CCL11, CCL13, CCL14, CCL16, Aplaviroc, Cenicriviroc (TAK-652, TBR-652), INCB009471, Maraviroc, Vicriviroc, PRO-140 antibody, CCL20, CCL19, CCL21, CCL1, CCL16, CCL25, CCL27, CCL28, CCL19, CCL21, CCL25, Bertilimumab, Carlumab, CXCL6, Emoctakin, Interleukin-8 (CXCL8, GCP-1), Navarixin, Ladarixin, Reparixin (repertaxin), CXCL1 (MGSA), CXCL2, CXCL3, CXCL5, CXCL6, CXCL7, Emoctakin, Garnocestim, Interleukin-8 (CXCL8, GCP-1), Danirixin, Elubrixin, Navarixin, Ladarixin, Reparixin (repertaxin), CXCL4 (PF4), CXCL9 (MIG), CXCL10 (IP-10), CXCL11 (I-TAC), Iroplact, Eldelumab, MIF, SDF-1 (CXCL12), Ubiquitin, Mavorixafor, Plerixafor (AMD3100), Ulocuplumab, CXCL13, CXCL16, CXCL11 (I-TAC), SDF-1 (CXCL12), Plerixafor (AMD3100), Lymphotactin-α (XCL1), Lymphotactin-β (XCL2), Pateclizumab, Fractalkine (CX3CL1), CC(β) chemokines, Chemerin, Resolvin E1, ARA-290, Asialo erythropoietin, Carbamylated erythropoietin, CNTO-530, Darbepoetin alpha, Epoetin alpha, Epoetin beta, Epoetin delta, Epoetin epsilon, Epoetin gamma, Epoetin kappa, Epoetin omega, Epoetin theta, Epoetin zeta, Erythropoietin (EPO), Erythropoietin-Fc, Methoxy polyethylene glycol-epoetin beta (CERA/Mircera), Peginesatide, Pegol sihematide (EPO-018B), Filgrastim, Granulocyte colony-stimulating factor, Lenograstim, Leridistim, Lipegfilgrastim, Nartograstim, Pegfilgrastim, Pegnartograstim, Ecogramostim, Granulocyte macrophage colony-stimulating factor, Milodistim, Molgramostim, Regramostim, Sargramostim, Mavrilimumab, Namilumab, Otilimab, Cilmostim, Interleukin-34, Lanimostim, Macrophage colony-stimulating factor, Mirimostim, Agerafenib, Eltrombopag, Pegacaristim, Promegapoietin, Romiplostim, Thrombopoietin (THPO, MGDF), Albinterferon, Interferon alpha (interferon alpha, IFN-α), Interferon alpha (IFNA1, IFNA2, IFNA4, IFNA5, IFNA6, IFNA7, IFNA8, IFNA10, IFNA13, IFNA14, IFNA16, IFNA17, IFNA21), Interferon alpha 2a, Interferon alpha 2b, Interferon alpha n1, Interferon alphacon-1, Interferon alpha-n3, Interferon beta (IFN-β) (IFNB1, IFNB3), Interferon beta 1a, Interferon beta 1b, Interferon kappa (IFN-ε/κ/τ/ζ, IFNK), Interferon omega (IFN-ω, IFNW1), Peginterferon alpha-2a, Peginterferon alpha-2b, Anifrolumab, Faralimomab, MEDI-545, Rontalizumab, Sifalimumab, Bifarcept, Interferon gamma (IFN-7), Interferon gamma 1b, Emapalumab, Fontolizumab, Interleukin 1 (α, β), Mobenakin, Pifonakin, AF-12198, Anakinra, IL-1RA, Isunakinra, Canakinumab, Gevokizumab, Lutikizumab, Decoy receptors: Rilonacept (IL-1 Trap), Adargileukin alpha, Aldesleukin, Celmoleukin, Denileukin diftitox, Interleukin 2, Pegaldesleukin, Teceleukin, Tucotuzumab celmoleukin, Basiliximab, Daclizumab (dacliximab), Inolimomab, Daniplestim, Interleukin 3, Leridistim, Milodistim, Muplestim, Promegapoietin, Binetrakin, Interleukin 4, Interleukin 13, Pitrakinra, Dupilumab, Pascolizumab, Interleukin 5, YM-90709, Benralizumab, Mepolizumab, Reslizumab, TPI ASM8, Atexakin alpha, Interleukin 6, ARGX-109, Clazakizumab, Elsilimomab, mAb 1339, Olokizumab, Sarilumab, Siltuximab, Sirukumab, Tocilizumab, Levilimab: Interleukin 7, Interleukin 9, Enokizumab, Ilodecakin, Interleukin 10 (CSIF), Interleukin 11 (AGIF), Oprelvekin, Edodekin alpha, Interleukin 12, Briakinumab, Ustekinumab, Binetrakin, Cintredekin besudotox, Interleukin 4, Interleukin 13, Anrukinzumab, Lebrikizumab, Tralokinumab, ALT-803, Interleukin 15, Interleukin 17 (A, B, C, D, E (interleukin 25), Brodalumab, Ixekizumab, Perakizumab, Remtolumab, Secukinumab, Vunakizumab, Iboctadekin, Interleukin 18, Interleukin 37, Tadekinig, IL18BP, Interleukin 19, Interleukin 20, Interleukin 24, Fletikumab, Denenicokin, Interleukin 21, NNC0114-0005, NNC0114-0006, Interleukin 22, Fezakinumab, Interleukin 23 (SGRF), Brazikumab, Briakinumab, Guselkumab, Risankizumab, Tildrakizumab, Ustekinumab, Interleukin 27 (interleukin 30), Interferon λ4 (IFN-λ4), Interleukin 28 (A (IFN-λ2), B (IFN-λ3)), Interleukin-29 (IFN-λ1), Interleukin 31, Interleukin 33, Interleukin 36 (α, β, γ), Interleukin 38, IL-36RA, Interleukin 14 (taxilin alpha, HMW-BCGF), Interleukin 16, Interleukin 24, Interleukin 26, Interleukin 32, Interleukin 34, Interleukin 35, Efavaleukin alpha, Efineptakin alpha, Activin (A, B, AB), Avotermin, BMP (10), Cetermin, GDF (2 (BMP9)), TGFβ (1, 2, 3): DMH-1, DMH-2, Dorsomorphin (BML-275), K-02288, ML-347 (LDN-193719, VU0469381), Ascrinvacumab, K-02288, ML-347 (LDN-193719, VU0469381), Dalantercept, Disitertide, Activin (A, B, AB), AMH (MIS), Avotermin, BMP (5, 6, 7, 8A, 8B), Eptotermin alpha, TGFβ (1, 2, 3), AMH (MIS), BMP (2, 4, 5, 6, 7, 8A, 8B), Dibotermin alpha, Eptotermin alpha, DMH-2, Dorsomorphin (BML-275), K-02288, Activin (A, B, AB), GDF (1, 3, 11 (BMP11)), Myostatin (GDF8), Nodal, Inhibin (A, B), Lefty (1, 2), A 83-01, SB-431542, SB-505124, Avotermin, GDF (10 (BMP3B), 11 (BMP11)), TGFβ (1, 2, 3), Fresolimumab, Lerdelimumab, Metelimumab, A 83-01, D-4476, GW-788388, LY-364947, LY-2109761, Galunisertib (LY-2157299), R-268712, RepSox (E-616452, SJN-2511), SB-431542, SB-505124, SB-525334, SD-208, BMP (2, 4, 5, 6, 7, 8A, 8B, 15, GDF9B)), BAMBI, Cerberus (CER1), Chordin, DAN (PARN), Decorin, Follistatin, Gremlin (Drm), LTBP1, Noggin, TGIF, Thrombospondin 1 (THBS1), tomoregulin 1, stamulumab, TRC105, endoglin, lymphotoxin, baminercept, plusonermin, sonermin, tasonfermin, afelimomab, certolizumab pegol, golimumab, infliximab, nerelimomab, ozoralizumab, remtolumab, placulumab, belimumab, brentuximab vedotin, conatumumab, dacetuzumab, denosumab, drozitumab, enavatuzumab, iratumumab, lexatumumab, lucatumumab, mapatumumab, oxelumab, ruplizumab, tabalumab, tavolixizumab, teneliximab, tigatuzumab, toralizumab, urelumab, utomilumab, varlilumab, vorsetuzumab, vorsetuzumab mafodotin, abrocitinib, baricitinib. filgotinib. momelotinib, oclacitinib, peficitinib, ruxolitinib, tofacitinib, upadacitinib, atiprimod, AZD-1480, baricitinib, CHZ868, cucurbitacin I (elatericin B, JSI-124), CYT387, Lestaurtinib, NSC-7908, NSC-33994, pacritinib, peficitinib, ruxolitinib, SD-1008, tofacitinib, cercosporamide, decernotinib (VX-509), peficitinib, TCS-21311, tofacitinib, WHI-P 154, ZM-39923, ZM-449829, cardiotrophin 1 (CT-1), FMS-like tyrosine kinase 3 ligand (FLT3L), leukemia/leukocyte inhibitory factor (LIF), Oncostatin M (OSM), thymic stromal lymphopoietin (TSLP), lestaurtinib, midostaurin, quizartinib, sorafenib, and sunitinib.

25. (canceled)

26. A method of claim 1 wherein the subject is suffering from a psychotic disorder, sleep disorder, depression, anxiety, panic disorder, obsessive-compulsive disorder, post-traumatic stress disorder, substance use disorder, pain, eating disorder, dependency or acute toxicity associated with narcotic agents such as LSD or MDMA, and or hot flushes associated with the menopause.

27. The method of claim 26 wherein 1) the subject is identified as suffering from a psychotic disorder, sleep disorder, depression, anxiety, panic disorder, obsessive-compulsive disorder, post-traumatic stress disorder, substance use disorder, pain, eating disorder, dependency or acute toxicity associated with narcotic agents such as LSD or MDMA, and or hot flushes associated with the menopause and 2) the (i) one or more or 5HT2A receptor agonists, and (ii) one or more 5HTA receptor modulator compounds are administered to the identified subject.

28. A method of claim 1 wherein the subject is suffering from hallucinogenic symptoms or occurrences.

29. (canceled)

30. A method of claim 1 wherein the subject is suffering from or susceptible to treatment resistant depression.

31. (canceled)

32. A method of claim 1 wherein the subject is suffering from post-traumatic stress disorder.

33. (canceled)

34. A method of claim 1 wherein the subject is suffering from or susceptible to substance use disorder.

35. (canceled)

36. A method of claim 1 wherein the subject is suffering from psychiatric disorder related sleep disturbances.

37-40. (canceled)
